(19) United States
(12) Patent Application Publication
(10) Pub. No.: US 2025/0064993 A1
(43) Pub. Date: Feb. 27, 2025

(54) NOVEL BISPIDINE-BASED METAL CHELATING LIGANDS DISPLAYING GOOD RELAXIVITY

(71) Applicants: UNIVERSITE DE STRASBOURG, STRASBOURG (FR); CENTRE NATIONAL DE LA RECHERCHE SCIENTIFIQUE, PARIS (FR)

(72) Inventors: Loïc CHARBONNIERE, WEYERSHEIM (FR); Maryame SY, CHOISY-LE-ROI (FR); Eva JAKAB TÓTH, OLIVET (FR); Aline NONAT, ECKBOLSHEIM (FR)

(73) Assignees: UNIVERSITE DE STRASBOURG, STRASBOURG (FR); CENTRE NATIONAL DE LA RECHERCHE SCIENTIFIQUE, PARIS (FR)

(21) Appl. No.: 18/724,773

(22) PCT Filed: Dec. 23, 2022
(86) PCT No.: PCT/EP2022/087686

§ 371 Date: Jun. 27, 2024

(30) Foreign Application Priority Data
Dec. 31, 2021 (EP) 21306966.9

(51) Int. Cl. A61K 51/04; C07D 471/08

(52) U.S. Cl. CPC A61K 51/0482; C07D 471/08

(57) ABSTRACT

Novel bispidine-based compounds including at least an optionally substituted ethanoic acid moiety at the N7-position of the bispidine scaffold, these bispidine-based compounds representing metal chelating ligands able to form metallic complexes having good properties in terms of relaxivity and stability, to complexes including a metal ion complexed with these bispidine-based compounds, and to the use of these bispidine-based compounds in the field of medical imaging or therapy, and more specifically MRI (magnetic resonance imaging) contrast agents and/or nuclear imaging agents for PET (positron emission tomography) and/or SPECT (single photon emission tomography).

### FIELD

The present invention relates to novel bispidine-based compounds comprising at least an optionally substituted ethanoic acid moiety at the N7-position of the bispidine scaffold, said bispidine-based compounds representing metal chelating ligands able to form metallic complexes having good properties in terms of relaxivity and stability, to complexes comprising a metal ion complexed with said bispidine-based compounds, and to the use of said bispidine-based compounds in the field of medical imaging or therapy, and more specifically as MRI (magnetic resonance imaging) contrast agents and/or nuclear imaging agents for PET (positron emission tomography) or SPECT (single photon emission tomography).

### BACKGROUND

Gadolinium-complexes have been used in millions of human examinations with magnetic resonance imaging (MRI) and are considered among the safest diagnostic drugs. The following gadolinium-based contrast agents (GBCAs) have been approved for clinical use: gadopentetate dimeglumine or [Gd(OH₂) (dtpa)]²-(Magnevist®, compound “a” in Scheme 1 below), gadoteridol (ProHance®), gadodiamide or [Gd(OH₂) (dtpa-bma)](Omniscan®, compound “b” in Scheme 1 below), gadoterate meglumine or [Gd(OH₂) (dota)]-(Dotarem®, compound “c” in Scheme 1 below), gadobutrol or [Gd(OH₂) (do3a-butrol)](Gadovist®, compound “d” in Scheme 1 below), gadoversetamide (OptiMARK®), gadoxetic acid (Primovist®), gadobenate dimeglumine or [Gd(OH₂) (bopta)]²-(MultiHance®, compound e in Scheme 1 below), and gadofosveset trisodium (Vasovist®/Ablavar®).

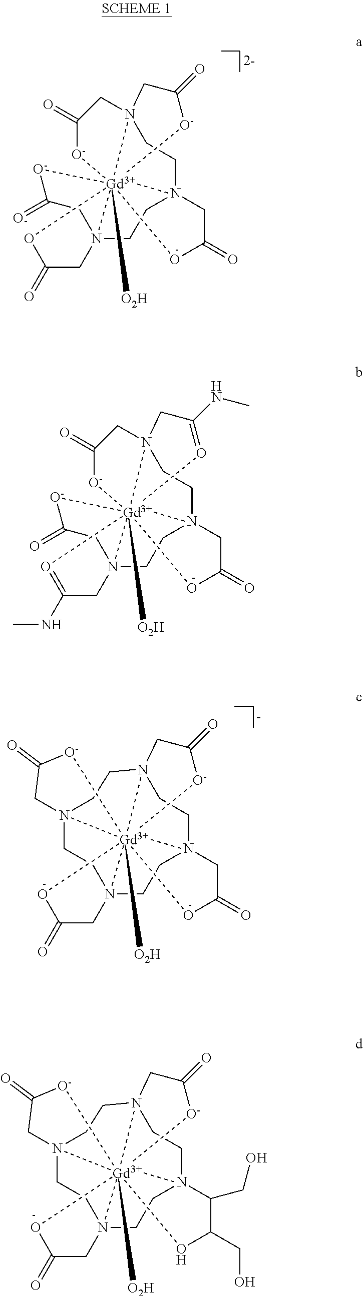

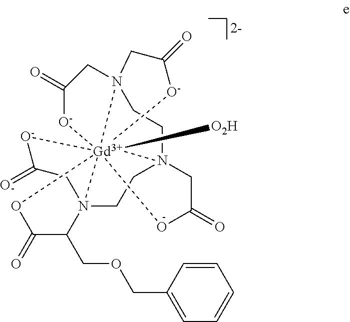

However, the recent emergence of nephrogenic systemic fibrosis and its causal link to Gd exposure, as well as the evidence on brain and bone accumulation of Gd have alerted the medical community. Nephrogenic systemic fibrosis (NSF) is a rare and serious syndrome that is associated with the exposure to GBCAs in patients with chronic kidney disease. NSF involves fibrotic changes in the skin and many organs.

Therefore, in 2010, the U.S. Food and Drug Administration (FDA) published revised labeling recommendations for four linear GBCAs which have been principally implicated in NSF, including gadodiamide (Omniscan®), gadobenate dimeglumine (MultiHance®), gadopentetate dimeglumine (Magnevist®), and gadoversetamide (OptiMARK®). In 2017, the European Medicines Agency has decided to stop marketing linear GBCAs contrast agents.

Furthermore, the negative outcome of using high quantities of Gd(III) based contrast agents, e.g. gadolinium accumulating in surface waters and coming from clinical waste waters, also arises an increasing environmental problem.

In this context, providing novel Gd(III) based contrast agents that can be administered in lower quantities and/or that are more stable as well as replacing Gd(III) with more biocompatible, safer paramagnetic metal ions have become major objectives.

Indeed, due to the low sensitivity of MRI as an imaging technique, large quantities of a contrast agent, often on the gram scale, must be injected into the patient to obtain useful images. The ability to reduce the quantity of GBCAs required is highly desirable, especially when considering the toxicity problems discussed above. One way in which the amount of contrast agent required can be reduced is to enhance its relaxivity. Relaxivity is the ability of the metal chelate to relax water protons, and is defined as the change in the relaxation rate of water divided by the millimolar concentration of the chelate. High relaxivities are indicative of more effective agents. All the commercially available GBCAs are very similar to each other in terms of relaxivity. As an example, Dotarem® displays a relaxivity r₁ of 3.7 mM⁻¹.s⁻¹ at pH of 7.4, 60 MHz, and 25° C.

Another way to reduce or avoid the risks of health problems associated with Gd³⁺ release is to provide stable GBCAs. Gd³⁺ release by GBCAs can be characterised by thermodynamic stability kinetic inertness. Thermodynamic stability refers to the Gibbs free energy involved in the complexation reaction and is defined by the stability constant log K (also called log K_{adL}, log Kₜₕₑᵣₘ and log Kₛₜ). Kinetic inertness refers to complex dissociation rate and is mainly reported as t_{1/2}, where t_{1/2} is defined as the time required for half of the GBCA's dissociation.

Research is also focused on providing alternative gadolinium (III)-free metal complexes. For example, US2020/157099 A1 describes Mn(II), Fe(II), Fe(III), Co(II) and Ni(II) ion macrocycle-based complexes to apply as MRI contrast agents and 52Mn-based PET diagnostic. The compound corresponding to the following formula:

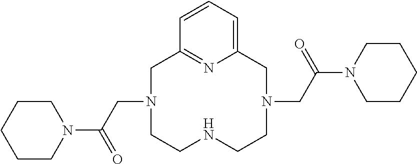 [SMILES: O=C(CN1CCNCCN(CC(=O)N2CCCCC2)Cc2cccc(n2)C1)N1CCCCC1]

is complexed with Mn(II) so as to form a Mn(III) macrocycle metal complexe [Mn (tPC2AM^{PYP})]²⁺ having a relaxivity r₁ of 4.90 mM⁻¹.s⁻¹ at pH of 7.4 and 25° C. and an half-life t_{1/2} of complex dissociation calculated at physiologic pH of 352 hours. However, the relaxivity as well as kinetic interness are not completely satisfactory.

Mangafodipir trisodium (Mn-DPDP), a contrast agent free of Gd(III), was marketed in the 1990's under the tradename Teslascan® comprising Mn(II) ion as the central paramagnetic ion. However, it was withdrawn from the US market in 2003 and from the EU market in 2010 due to low sales, poor clinical performance, and concerns over toxicity.

Accordingly, there is a need of novel metal chelating ligands able to form complexes with other metals than gadolinium (III) and which have improved relaxivity while guaranteeing good kinetic inertness.

### SUMMARY

Thus, the aim of the present invention is to overcome the drawbacks of the cited prior art, and more particularly, to provide novel metal chelating ligands having good relaxation properties, low toxicity and/or which can be easily eliminated by renal route, water solubility and improved kinetic inertness and/or low dissociation rate.

A first object of the present invention is a bispidine-based metal chelating ligand corresponding to the following formula (I):

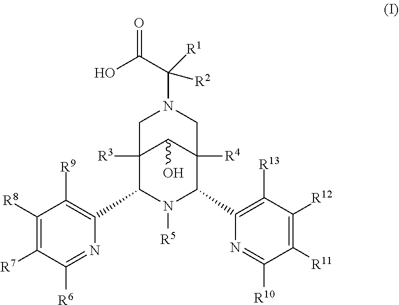

in which:
* R¹ represents an hydrogen atom or a C₁-C₈ alkyl group,
* R² represents an hydrogen atom, a C₁-C₈ alkyl group, a group corresponding to formula (II): —(CH₂)ₙ—NH₂ (II), where 3≤n≤18, or a group corresponding to the following formula (III):

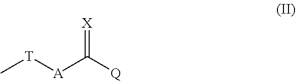

where:
* T represents a C₁-C₁₇ alkylene group, a C₁-C₁₇ alkenylene group, or a C₁-C₁₇ alkynylene group,
* A represents —CH₂— or —NH—,
* X represents an oxygen atom, a sulfur atom, or a NH group, and
* Q represents:
   an alkyl group optionally substituted with a functional group,
   a NHR¹⁴ group where R¹⁴ represents an hydrogen atom, or an aryl group optionally substituted with a functional group,
a polyethylene glycol group corresponding to formula (IV): —CH₂—(CH₂—O—CH₂)_{q}—CH₂OH(IV), where 1≤q≤24, or
an OH group,
* R³ and R⁴, which may be identical or different, and preferably identical, represent a CH₂OH group, a CO₂H group, or a CONHR¹⁵ group, where R¹⁵ represents:
   an alkyl group optionally substituted with a functional group,
   an aryl group optionally substituted with a functional group, or
   a polyethylene glycol group corresponding to formula (V): —CH₂—(CH₂—O—CH₂)ᵣ—CH₂OH(V), where 1≤r≤24,
* R⁵ represents an hydrogen atom, an alkyl group, a group of formula (VI): —(CH₂)ₘ—CO₂H(VI) where 1≤m≤5, a polyethylene glycol group corresponding to formula (VII): —CH₂—(CH₂—O—CH₂)ₛ—CH₂OH(VII), where 1≤s≤18, or a group of formula (III) as defined above,
* R⁶, R⁷, R⁸, R⁹, R¹⁰, R¹¹, R¹², and R¹³, which may be identical or different, represent a hydrogen atom, an OH group, an ether group OR¹⁶ where R¹⁶ is an alkyl group, a CO₂H group, or a CONHR¹⁷ group, where R¹⁷ represents an alkyl group optionally substituted with a functional group,
with the proviso that:
   when R² represents a group corresponding to formula (II): —(CH₂)ₙ—NH₂ (II), and R⁵ represents an alkyl group, then R³ and R⁴ are different from CO₂H groups,
   when R⁵ represents an alkyl group, then R¹ and R² are different from hydrogen atoms, and
   when R² represents a group of said formula (III), where T represents a C₁-C₁₇ alkylene group, A represents —NH—, X represents an oxygen atom, and Q represents a C₁-C₅ alkyl group substituted with a functional group, then R³ and R⁴ are different from CO₂H groups.

Thus, the ligands of the invention can lead to metal complexes having good relaxation properties, low toxicity and/or which can be easily eliminated by renal route, water solubility and improved kinetic inertness and/or low dissociation rate.

### BRIEF DESCRIPTION OF THE FIGURE

FIG. 1 shows the bispidine-based metal chelating ligand corresponding to the formula (I).

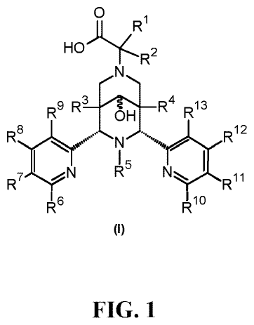

DETAILED DESCRIPTION Bispidines are chelators based on a 3,7-diazabicyclo[3.3.1]nonane scaffold. The bispidine scaffold is formed by two fused cyclohexylamine rings.

More particularly, the metal chelating ligand (I) of the present invention has a so-called 2,4-disubstituted bispidol core and its IUPAC numbering is presented as follows:

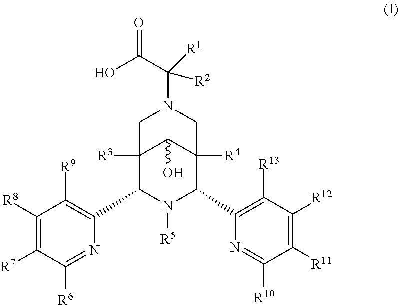

Said metal chelating ligand (I) is expected to coordinate at least in a fivedentate manner, involving two pyridine and two bispidine nitrogens as well as the methylene carboxylate at the N7 position and appropriate R¹ and R² groups on the methylene moiety.

The bispidine-based metal chelating ligands of the present invention are highly preorganized ligands that can accommodate metal ions with cis-octahedral, square-pyramidal, or pentagonal geometries. The bispidine-based metal chelating ligands of the invention form thermodynamically very stable metal complexes with transition-metal ions which show high kinetic inertness. Modification of the coordinating pendant arms can be used to tune the ligand denticity as well as all electronic, thermodynamic, and kinetic parameters such as the ligand field, the metal selectivity, and the stability constants. Such properties are very appealing for applications in diagnosis as chelators for metals other than gadolinium, and more specifically for manganese and copper metals, as well as radiometals such as ⁶⁴Cu.

In the present invention, the expression “when R⁵ represents an alkyl group, then R¹ and R² are different from hydrogen atoms” means that when R⁵ represents an alkyl group, then the pair (R¹, R²) is different from the pair (hydrogen atom, hydrogen atom). In other words, when R⁵ represents an alkyl group, then R¹ and R² are different from hydrogen atoms at the same time or together. Therefore, when R⁵ is an alkyl group, the couple or pair (R¹, R²) can be a couple (R¹=hydrogen atom, R²=something else than an hydrogen atom) or (R¹=something else than an hydrogen atom, R²=hydrogen atom) or (R¹=something else than an hydrogen atom, R²=something else than an hydrogen atom).

### The R¹ Group

The R¹ group represents an hydrogen atom or a C₁-C₈ alkyl group.

The C₁-C₈ alkyl group as the R¹ group is preferably a linear C₁-C₅ alkyl group, and more preferably a methyl group (i.e. C₁ alkyl group).

In one preferred embodiment, the R¹ group represents an hydrogen atom.

### The R² Group

R² represents an hydrogen atom, a C₁-C₈ alkyl group, a group corresponding to formula (II) as defined in the present invention or a group corresponding to formula (III) as defined in the present invention.

The C₁-C₅ alkyl group as the R² group is preferably a linear C₁-C₅ alkyl group, and more preferably a methyl group.

The group corresponding to formula (II): —(CH₂)ₙ—NH₂ (II), where 3≤n≤18 as the R² group is preferably such that 4≤n≤15, and more preferably such that 5≤n≤10.

The group corresponding to the formula (III) as defined in the present invention as the R² group is defined by the groups T, A, X and Q.

T represents a C₁-C₁₇ alkylene group, a C₁-C₁₇ alkenylene group, or a C₁-C₁₇ alkynylene group, and preferably a C₁-C₁₇ alkylene group.

An alkylene group is a bivalent saturated aliphatic hydrocarbon radical.

The C₁-C₁₇ alkylene group can be a branched or a linear alkylene group, and preferably a linear alkylene group. The alkylene group is preferably a C₁-C₁₀ alkylene group, and more preferably a C₁-C₆ alkylene group.

An alkenylene group is a bivalent insaturated aliphatic hydrocarbon radical comprising one or more double bonds C═C.

The C₁-C₁₇ alkenylene group can be a branched or linear (straight-chain)alkenylene group, and preferably a linear alkenylene group. The alkenylene group is preferably a C₂-C₆ alkenylene group, and more preferably a C₂-C₄ alkenylene group.

An alkynylene group is a bivalent insaturated aliphatic hydrocarbon radical comprising one or more triple bonds C≡C.

The C₁-C₁₇ alkynylene group can be a branched or linear (straight-chain)alkynylene group, and preferably a linear alkynylene group. The alkynylene group is preferably a C₂-C₆ alkynylene group, and more preferably a C₂-C₄ alkynylene group.

According to one particularly preferred embodiment, at least one of R¹ ou R² group is different from an hydrogen atom, and advantageously, the group R² is different from an hydrogen atom (in other word, the group R² represents advantageously an C₁-C₈ alkyl group, a group corresponding to formula (II) or a group corresponding to the following formula (III)).

### The Q Group

The alkyl group optionally substituted with a functional group as a Q group can be a linear or a branched alkyl group, and preferably a linear alkyl group.

The alkyl group optionally substituted with a functional group as a Q group can be a non-susbtituted C₁-C₄ alkyl group or an alkyl group comprising at least 5 carbon atoms and being optionally substituted with a functional group.

The alkyl group optionally substituted with a functional group as a Q group is preferably a C₅-C₂₀ alkyl group optionally substituted with a functional group, and more preferably a C₆-C₁₈ alkyl group optionally substituted with a functional group.

The NHR¹⁴ group as the Q group is preferably such that R¹⁴ represents an aryl group optionally substituted with a functional group, said functional group being as defined in the present invention.

The aryl group as R¹⁴ can be a C₅-C₂₀ aryl group optionally substituted with a functional group, and preferably a C₆-C₁₂ aryl group optionally substituted with a functional group.

The aryl group can be a phenyl group, a pyridine, a pyrimidine, a triazine, a thiophene, a porphyrine, an aryl-based cyanine or another aryl-based dye, and preferably a phenyl group.

The phenyl group can be substituted with one or more W groups (W being different from the functional group) selected from hydroxyl, ether, primary and secondary amine, carboxylic acid, amide, nitro, and thiol groups.

The polyethylene glycol group corresponding to formula (IV): —CH₂—(CH₂—O—CH₂)_{q}—CH₂OH(IV), where 1≤q≤24, as the Q group is preferably such that 1≤q≤12 and more preferably such that 1≤q≤4.

In one preferred embodiment, R² represents a group corresponding to formula (II) or (III) as defined in the present invention, and more preferably a group corresponding to formula (III).

Indeed, the introduction of a group of formula (II) or (III) at the N7 position of the bispidine scaffold enables the formation of Mn(II) complexes with a sufficient thermodynamic stability and an extremely high kinetic inertness to be used as MRI contrast agents and allows to modulate the physicochemical properties of the bispidine and its associated metal complexes, as well as their biodistribution and biotoxicological properties.

The following embodiments as a group of formula (III) are particularly advantageous:
A represents —NH—, X represents an oxygen atom, and Q represents an alkyl group optionally substituted with a functional group or a polyethylene glycol group corresponding said formula (IV);
A represents —NH—, X represents a sulfur atom, and Q represents a NHR¹⁴ group where R¹⁴ represents an aryl group optionally substituted with a functional group; or
A represents —CH₂—, X represents an oxygen atom, and Q represents an OH group.
   The R³ and R⁴ Groups

R³ and R⁴, which may be identical or different, represent a CH₂OH group, a CO₂H group, or a CONHR¹⁵ group, and preferably a CH₂OH group or a CO₂H group.

The alkyl group optionally substituted with a functional group as R¹⁵ can be a linear or a branched alkyl group, and preferably a linear alkyl group.

The alkyl group optionally substituted with a functional group as R¹⁵ is preferably a C₅-C₂₀ alkyl group optionally substituted with a functional group, and more preferably a C₆-C₁₈ alkyl group optionally substituted with a functional group.

The functional group is as defined in the present invention.

The aryl group optionally substituted with a functional group as R¹⁵ can be selected from a C₅-C₂₀ aryl group optionally substituted with a functional group, and preferably a C₆-C₁₂ aryl group optionally substituted with a functional group.

The aryl group as R¹⁵ can be a phenyl group, a pyridine, a pyrimidine, a triazine, a thiophene, a porphyrine, an aryl-based cyanine, and another aryl-based dye, and preferably a phenyl group.

The phenyl group can be substituted with one or more W groups selected from hydroxyl, ether, primary and secondary amine, carboxylic acid, amide, nitro, and thiol groups.

The polyethylene glycol group corresponding to formula (V): —CH₂—(CH₂—O—CH₂)ᵣ—CH₂OH(V), where 1≤r≤24 as R¹⁵ is preferably such that 1≤r≤12.

### The R⁵ Group

R⁵ represents an hydrogen atom, an alkyl group, a group of formula (VI): —(CH₂)ₘ—CO₂H(VI) where 1≤m≤5, a polyethylene glycol group corresponding to formula (VII): —CH₂—(CH₂—O—CH₂)ₛ—CH₂OH(VII), where 1≤s≤18, or a group of formula (III) as defined above.

The alkyl group as R⁵ can be a linear or a branched alkyl group, and preferably a linear alkyl group.

The alkyl group as R⁵ is preferably a C₁-C₅ alkyl group, and more preferably a methyl group.

The group of formula (VI):—(CH₂)ₘ—CO₂H(VI) where 1≤m≤5 as R⁵ is preferably such that 1≤m≤3.

The polyethylene glycol group corresponding to formula (VII): —CH₂—(CH₂—O—CH₂)ₛ—CH₂OH(VII), where 1≤s≤18 as R⁵ is preferably such that 1≤ S≤10.

The group of formula (III) as R⁵ is as defined in the present invention for R²

In one preferred embodiment, R⁵ represents an alkyl group or a group of formula (VI):—(CH₂)ₘ—CO₂H(VI) where 1≤m≤5.

The R⁶, R⁷, R⁸, R⁹, R¹⁰, R¹¹, R¹², and R¹³ groups

The alkyl group as R¹⁶ can be a linear or a branched alkyl group, and preferably a linear alkyl group.

The alkyl group as R¹⁶ is preferably a C₁-C₁₀ alkyl group, and more preferably a C₁-C₅ alkyl group, and even more preferably a methyl or ethyl group.

The alkyl group optionally substituted with a functional group as R¹⁷ can be a linear or a branched alkyl group, and preferably a linear alkyl group.

The alkyl group optionally substituted with a functional group as R¹⁷ is preferably a C₅-C₂₀ alkyl group optionally substituted with a functional group, and more preferably a C₆-C₁₈ alkyl group optionally substituted with a functional group.

The functional group is as defined in the present invention.

The aryl group optionally substituted with a functional group as R¹⁷ can be selected from a C₅-C₂₀ aryl group optionally substituted with a functional group, and preferably a C₆-C₁₂ aryl group optionally substituted with a functional group.

The aryl group as R¹⁷ can be a phenyl group, a pyridine, a pyrimidine, a triazine, a thiophene, a porphyrine, an aryl-based cyanine, and another aryl-based dye, and preferably a phenyl group.

The phenyl group can be substituted with one or more W groups (W being different from the functional group) selected from hydroxyl, ether, primary and secondary amine, carboxylic acid, amide, nitro, and thiol groups.

In one preferred embodiment, R⁶, R⁷, R⁸, R⁹, R¹⁰, R¹¹, R¹², and R¹³ groups represent hydrogen atoms or at least R⁷, R⁹, R¹¹, and R¹³ represent hydrogen atoms. In other words, the pyridine rings can be substituted in 4 and/or 6 positions (i.e. at least one of R⁶, R⁸, R¹⁰, and R¹² is different from an hydrogen atom).

In a preferred embodiment, which can be advantageously combined with the preceding preferred embodiment, R⁶ and R¹⁰ are identical, R⁷ and R¹¹ are identical, R⁸ and R¹² are identical, and R⁹ and R₁₃ are identical.

### The Functional Group

The functional group can be selected from the following groups: amine, amide, sulfonate, sulfate, quaternaty ammonium, hydroxyl, phosphonate, succinimidyl ester, sulfosuccinimidyl ester, isothiocyanate, isocyanate, iodoacetamide, maleimide, sulfonyl halide, acid halide (e.g. preferably acid chloride and acid bromide), carbodiimide, biotin, azido, alkyne, and a —C(O)—Z group, where Z is selected from an —OH, —N-glycine, —N-lysine, —Y-L-NH₂, —Y-L-COOH and —Y-L-SH group, where Y is selected from N and O atoms, and L is selected from a C₁-C₁₇ alkylene group, a C₁-C₁₇ alkenylene group, a C₁-C₁₇ alkynylene group, and a phenylene group.

The C₁-C₁₇ alkylene, C₁-C₁₇ alkenylene, and C₁-C₁₇ alkynylene groups are as defined in the present invention.

The phenylene group can be substituted with one or more W groups selected from hydroxyl, ether, primary and secondary amine, carboxylic acid, amide, nitro, and thiol group.

The functional group can either provide solubility, lipophilicity or improved relaxivity (e.g. amide, quaternary ammonium).

It can also be a recognition function which can interact with biological molecules of interest (e.g. biotin which interact with streptavidin or aromatic compounds with human serum albumin (HSA)) or a reactive function which is capable of reacting with a biological molecule to form a target molecule that provides a specific affinity for a given compound in a medium. Applications of such target molecules in the field of MRI are blood-pool agents (BPAs) for magnetic resonance angiography which remain confined to the intravascular space due to their high affinity with the seric protein HAS or molecular resonance (MR) contrast agents which target a specific protein, cell type or biological process. Application of such target molecules in the field of nuclear medicine are peptide-conjugates or immunoradiotracors for immunotherapy and immuno-PET imaging. Such agents are specifically directed to the cell/protein of interest and have revolutionized treatment strategies for several types of cancer.

The biological molecule can be a sugar, a peptide, a protein, a nucleotide, a nanobody (V_{HH}), or an antibody.

When Q of R² represents an alkyl group optionally substituted with a functional group, said functional group is preferably selected from hydroxyl, amine, quaternary ammonium, maleimide, succinimidyl ester, and sulfosuccinimidyl ester.

When R¹⁴ (for Q) of R² represents an aryl group optionally substituted with a functional group, said functional group is preferably selected from isothiocyanate and isocyanate.

When Q of R⁵ represents an alkyl group optionally substituted with a functional group, said functional group is preferably selected from amide, succinimidyl ester and sulfosuccinimidyl ester.

When R¹⁴ (for Q) of R⁵ represents an aryl group optionally substituted with a functional group, said functional group is preferably isothiocyanate.

When R¹⁵ represents an alkyl group optionally substituted with a functional group, said functional group is preferably selected from amine, quaternary ammonium, maleimide, succinimidyl ester, and sulfosuccinimidyl ester.

When R¹⁵ represents an aryl group optionally substituted with a functional group, said functional group is preferably selected from isothiocyanate and isocyanate.

R¹⁷ represents preferably an alkyl group.

In one particularly preferred embodiment, the bispidine-based metal chelating ligand of the invention is preferably selected from the following formulae (I-a) to (I-h):

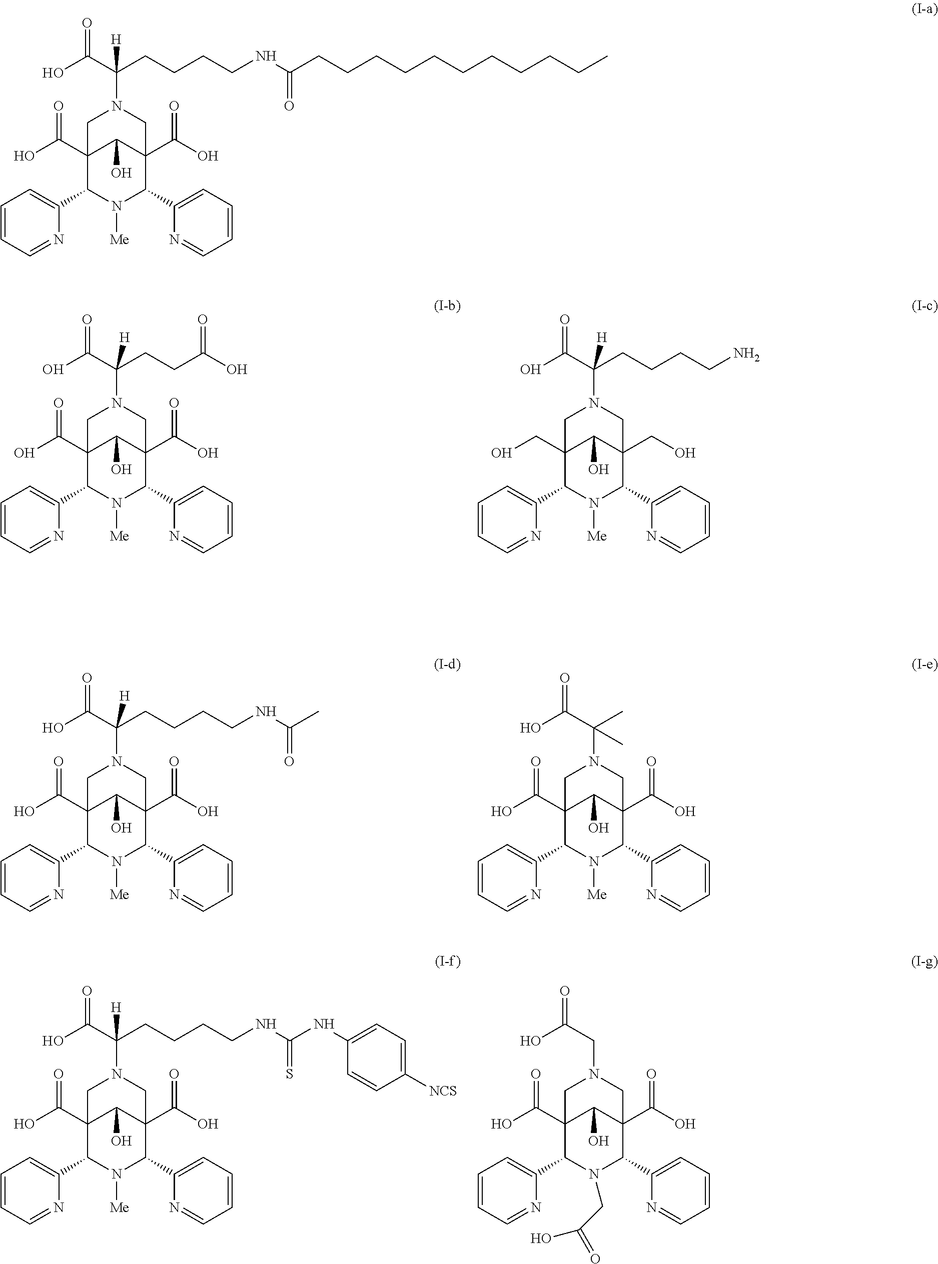

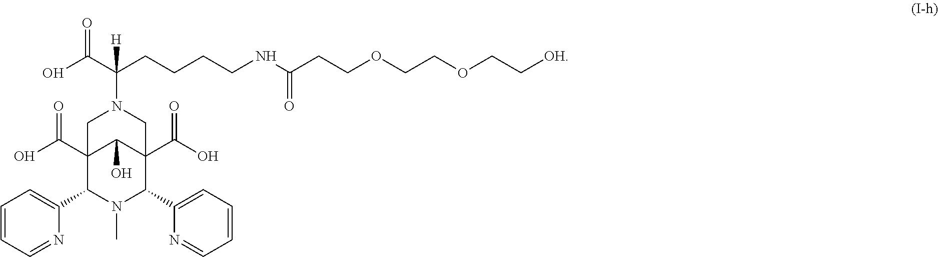 [SMILES: CN1[C@@H](c2ccccn2)C2(C(=O)O)CN([C@@H](CCCCNC(=O)CCOCCOCCO)C(=O)O)CC(C(=O)O)([C@H]2O)[C@H]1c1ccccn1]

A second object of the present invention is a complex of a metal M, wherein said complex comprises a metal ion of said metal M complexed with a bispidine-based metal chelating ligand (I) as defined in the first object of the present invention, said metal M being selected from copper, manganese, gallium, cobalt, zinc, nickel, and iron, and preferably selected from copper and manganese.

The metal M can be radioactive.

The metal ion can be selected from Mn²⁺, Mn³⁺, Cu²⁺, Cu⁺, Ga³⁺, Co²⁺, Co³⁺, Fe²⁺, Fe³⁺, Zn²⁺, or Ni²⁺.

The complexes of the present invention exhibit high relaxivity, show a favorable pharmacokinetic profile, are completely excreted, are chemically stable, exhibit high water solubility, offer the possibility of providing safe alternatives to traditional GBCAs and offer the potential for a significant dose reduction in comparison to state-of-the-art Mn-based CAs, and are suitable for imaging of different body regions.

A third object of the present invention is the use of a bispidine-based metal chelating ligand (I) as defined in the first object of the present invention or a complex as defined in the second object of the present invention, in the field of medical imaging or therapy, and more specifically as MRI (magnetic resonance imaging) contrast agents and/or nuclear imaging agents for PET (positron emission (single photon emission tomography).

Positron emission tomography (PET) is a highly sensitive imaging technique with high tissue penetration. This technique can allow for the in vivo imaging of diseased tissues by targeting biochemical processes; thus allowing for detection of disease before physical changes occur.

More particularly, the bispidine-based metal chelating ligand (I) as defined in the first object of the present invention can be used as a chelator for radioactive copper for application in immuno-positron emission tomography (PET).

More particularly, the bispidine-based metal chelating ligand (I) as defined in the first object of the present invention can be used as bifunctional chelating agents for HER2+ immuno-imaging of breast cancer or for PET immuno-imaging of multiple myeloma, for radioimmunotherapy or again as a theranostic probe combining therapy and diagnostic.

The present invention is illustrated in more detail in the examples below, but it is not limited to said examples.

### EXAMPLES

Solvents and starting materials were purchased from Aldrich, Acros and Alfa Aesar and used without further purification. IR spectra were recorded on a Perkin Elmer Spectrum One Spectrophotometer as solid samples and only the most significant absorption bands are given in cm⁻¹. Elemental analyses and mass spectrometry analysis were carried out by the Service Commun d′Analyses of the University of Strasbourg. ¹H and ¹³C NMR spectra and 2D COSY, NOESY, HSQC, and HMBC experiments were recorded on Avance 300 and Avance 400 spectrometers operating at 7.04 T and 9.39 T, respectively. Chemical shifts are reported in ppm, with residual protonated solvent as internal reference.

### Example 1: Synthesis of Intermediate Compound 4

Compound 4 was prepared according to the following scheme 2:

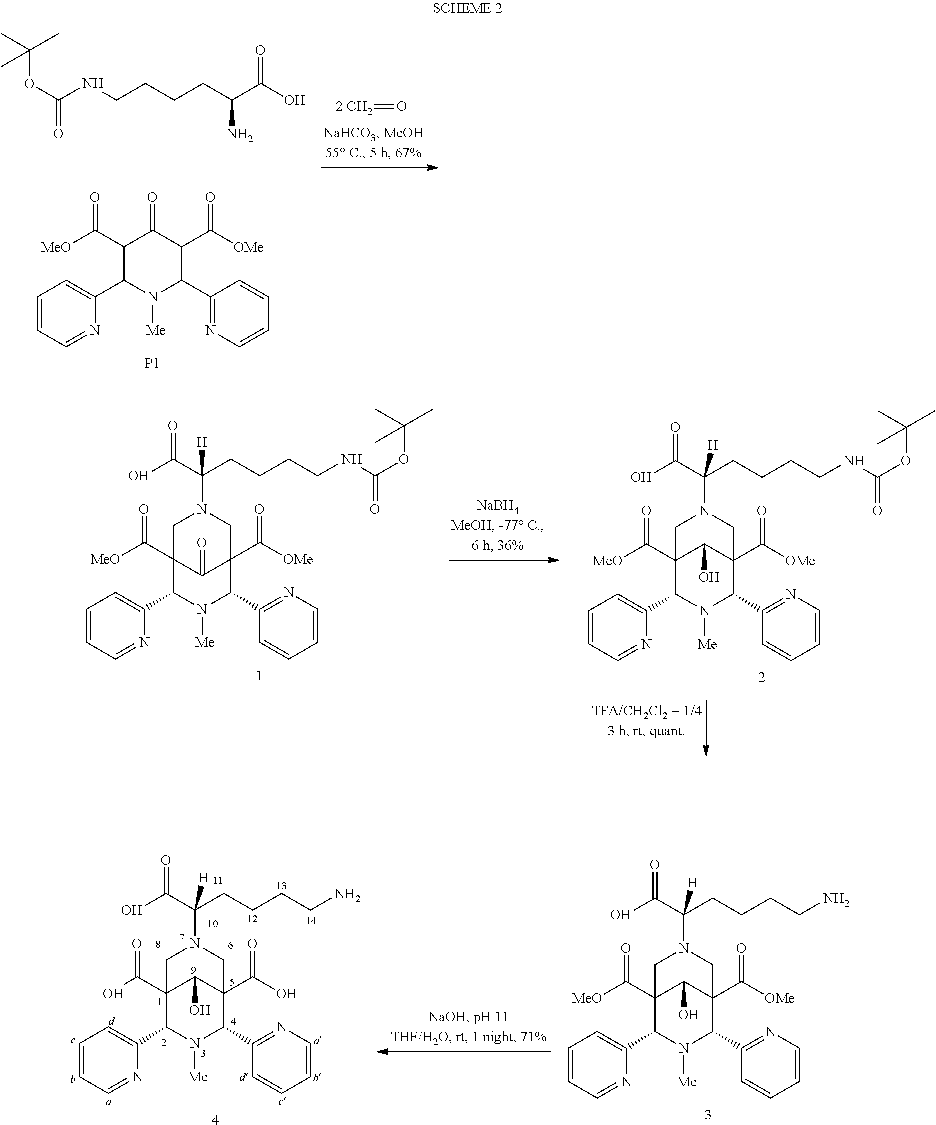

P1 is a well-known starting material to lead to bispidone and bispidine compounds.

Bispidone 1. In a solution of methanol (60 mL), NE-Boc-L-lysine (4.19 g, 17.40 mmol) and NaHCO₃ (1.46 g, 17.40 mmol) were mixed under stirring during 1 h at 45° C. Then, formaldehyde (3.5 mL, 47.50 mmol) and precursor P1 (6.06 g, 15.80 mmol) in methanol (20 mL) were added at room temperature (rt) and the mixture was stirred under reflux during 5 h. At the end of the reaction, the solution was filtered and solvents were removed under reduced pressure. The obtained solid was dissolved in a minimum of methanol and was precipitated upon dropwise addition in a large amount of diethyl ether. After centrifugation, precipitates were collected and dried to give a pure beige solid (6.93 g, 67%).

TLC (Al₂O₃; DCM/MeOH, 85/15); Rf=0.5.

¹H-NMR (400 MHZ, CDCl₃): δ 8.95 (m, 2H, Ha+Ha′), 7.70 (t, J=7.6 Hz, 1H, Hc′), 7.59 (t, J=7.5 Hz, 1H, Hc), 7.35 (m, 1H, Hb′), 7.17 (m, 3H, Hd′+Hd+Hb), 4.67 (s, 1H, H2), 4.56 (s, 1H, H4), 3.81 (broad s, 1H, COOH), 3.77 (s, 3H, OCH₃), 3.66 (s, 3H, OCH₃), 3.22 (AB system, δ_{A}=3.60, δ_{B}=2.84, J_{AB}=12.0 Hz, V_{A}=1440 Hz, V_{B}=1136 Hz, 2H, H8/H6), 2.96 (AB system, δ_{A}=3.29, δ_{B}=2.62, J_{AB}=12.0 HZ, V_{A}=1316 Hz, V_{B}=1048 Hz, 2H, H8/H6), 2.99 (m, 2H, H14), 2.57 (d, 1H, H10), 1.86 (s, 3H, CH₃), 1.64-1.22 (m, 15H, H11/H12/H13/OC(CH₃)₃).

¹³C-NMR (100 MHZ, CDCl₃): δ 202.7 (C₉), 177.0 (COOH), 168.0 (COOMe), 167.5 (COOMe), 156.1 (3C, 1Cpy+Cpy′+COOtBu), 151.5 (Ca/Ca′), 151.1 (Ca/Ca′), 137.5 (Cc/Cc′), 137.0 (Cc/Cc′), 124.5 (Cb/Cb′/Cd/Cd′), 124.2 (Cb/Cb′/Cd/Cd′), 124.0 (Cb/Cb′/Cd/Cd′), 121.7 (Cb/Cb′/Cd/Cd′), 78.7 (C(OCH₃)₃), 74.0 (C10), 72.6 (2C, C2+C4), 63.0 (2C, C1+C5), 57.5 (C6/C8), 54.7 (C6/C8), 52.8 (OCH₃), 52.5 (OCH₃), 43.0 (CH₃), 40.3 (C14), 29.9 (C11), 28.9 (C13), 28.5 (C(CH₃)₃), 24.0 (C12).

IR (cm⁻¹, ATR) v 3366 (broad, N—H amide), 2943, 2860 (broad, O—H acid), 1739 (s, C═O ester), 1695 (s, C═O acid), 1572 (s, C═C aromatic), 1254 (s, C—O).

Electrospray ionization ESI/MS⁺: m/z=654.31 ([M+H]⁺, 100%), 655.31 ([M+H]⁺, 35.7%), 656.32 ([M+H]⁺, 6.2%), 657.32 ([M+H]⁺, 0.7%).

Bispidol 2. Bispidone 1 (3.08 g, 4.71 mmol) was dissolved in anhydrous methanol (110 mL) and was cooled at −77° C. on a bath of acetone/dry ice. Then, sodium borohydride (240.00 mg, 7.07 mmol) was slowly added. After 6 h at −77° C., the mixture was placed at 4° C. for one night. Then NH₄Cl (520.00 mg, 9.72 mmol) was added in the flask and the mixture was stirred during 10 minutes. Solvents were removed under reduced pressure and the obtained solid was purified by flash chromatography column (reverse phase, CH₃CN/H₂O 20/80-25/75). The pure compound 2.2H₂O was obtained as a white solid (1.10 g, 32%).

TLC (SiO₂; DCM/MeOH, 80/20); Rf=0.3-0.5.

¹H-NMR (400 MHZ, MeOD): δ=8.67 (m, 2H, Ha+Ha′), 7.81 (m, 2H, Hc+Hc′), 7.60 (m, 2H, Hd+Hd′), 7.38 (m, 2H, Hb+Hb′), 4.89 (s, 2H, H2+H4), 4.50 (s, 1H, H9), 4.30 (AB system, δ_{A}=4.60, δ_{B}=4.00, J_{AB}=12.0 Hz, 2H, H6/H8), 3.96 (AB system, δ_{A}=4.10, δ_{B}=3.91, J_{AB}=12.0 Hz, 2H, H6/H8), 3.76 (m, 1H, H10), 3.63 (s, 3H, OCH₃), 3.55 (s, 3H, OCH₃), 3.08 (m, 2H, H14), 2.33-2.17 (m, 2H, H11), 1.73 (m, 4H, H12+H13), 1.65 (s, 3H, CH₃), 1.39 (s, 9H, C(CH₃)₃).

¹³C-NMR (75 MHZ, MeOD): § 172.8 (COOH), 170.4 (2C, COOMe), 158.3 (COOtBu/Cpy/Cpy′), 157.8 (COOtBu/Cpy/Cpy′), 157.7 (COOtBu/Cpy/Cpy′), 150.6 (Ca/Ca′), 150.1 (Ca/Ca′), 138.1 (Cc/Cc′), 138.0 (Cc/Cc′), 128.6 (2C, Cd+Cd′), 124.9 (Cb/Cb′), 124.8 (Cb/Cb′), 79.7 (C(CH₃)₃), 73.8 (C9), 72.0 (C10), 67.8 (C2/C4), 67.5 (C2/C4), 56.8 (C8/C6), 53.0 (OCH₃), 52.9 (OCH₃), 52.3 (2C, C1+C5), 51.2 (8/6), 41.6 (CH₃), 41.1 (C14), 30.7 (C13), 30.0 (C11), 28.7 (C(CH₃)₃), 25.1 (C12).

Electrospray ionization ESI/MS⁺: m/z=656.33 ([M+H]⁺, 100%), 657.34 ([M+H]⁺, 35.7%), 658.34 ([M+H]⁺, 3.5%).

Elemental analysis calculated for C₃₃H4509N5. 1.5H₂O: C, 58.05, H, 7.09, N, 10.26. Found: C, 57.82, H, 6.81, N, 10.38.

Bispidol 3. Bispidol 2 (413.0 mg, 0.74 mmol) was dissolved in CH₂Cl₂ (8 mL) and trifluoroacetic acid (2 mL) and the mixture was stirred at room temperature. After 3 h, solvents were evaporated under reduced pressure and the crude TFA salt was used in the next step without purification (413 mg).

TLC (C₁₈; CH₃CN/H₂O, 0.1% TFA, 50/50); Rf=0.8.

¹H-NMR (400 MHZ, MeOD): δ 8.76 (m, 2H, Ha+Ha′), 7.94 (m, 2H, Hc+Hc′), 7.77 (m, 2H, Hd+Hd′), 7.52 (m, 2H, Hb+Hb′), 5.63 (s, 1H, H2/H4), 5.61 (s, 1H, H2/H4), 4.41 (s, 1H, H9), 3.72 (s, 3H, OCH₃), 3.69 (s, 3H, OCH₃), 3.33 (AB system, δ_{A}=3.52, δ_{B}=3.13, J_{AB}=12.0 HZ, V_{A}=1408 Hz, V_{B}=1252 Hz, 2H, H6/H8), 3.39 (AB system, δ_{A}=3.41, δ_{B}=3.38, J_{AB}=10.5 Hz, V_{A}=1363 Hz, V_{B}=1353 Hz, 1H, H10), 3.13 (m, 2H, H6/H8), 3.03 (t, J=8.5 Hz, 2H, H14), 2.35-1.92 (m, 2H, H11), 2.31 (s, 3H, CH₃), 1.90 (m, 2H, H13), 1.61 (m, 2H, H12).

¹³CNMR (75 MHZ, D₂O): δ 173.6 (COOH), 170.5 (COOMe), 170.4 (COOMe), 152.9 (Cpy/Cpy′), 152.8 (Cpy/Cpy′), 151.0 (Ca/Ca′), 150.4 (Ca/Ca′), 139.1 (Cc/Cc′), 139.0 (Cc/Cc′), 129.0 (Cd/Cd′), 128.6 (Cd/Cd′), 126.2 (Cb/Cb′), 126.1 (Cb/Cb′), 73.9 (C9), 68.1 (C10), 67.6 (C2/C4), 67.1 (C2/C4), 55.8 (C6/C8), 54.4 (C1/C5), 53.7 (C1/C5), 53.3 (OCH₃), 53.2 (OCH₃), 50.4 (C6/C8), 43.4 (CH₃), 40.7 (C14), 29.8 (C11), 28.8 (C13), 25.0 (C12).

Compound 4. Bispidol 3 (279.0 mg, 0.50 mmol) was dissolved in a mixture of H₂O/THF (8 mL/2 mL) and sodium hydroxide (240.0 mg, 3.0 mmol) was added at room temperature under stirring. After 48 h, the mixture was evaporated under reduced pressure and was purified by column chromatography (reverse phase, 100% H₂O to 100% MeOH in 15 min).

Pure compound 4.2Na (265.0 mg, 71%) was obtained as a white powder.

TLC (C₁₈; CH₃CN/H₂O, 20/80), Rf=0.4.

¹H-NMR (400 MHZ, D₂O): δ 8.48 (m, 2H, Ha+Ha′), 7.54 (m, 2H, Hc+Hc′), 7.30 (d, J=9.0 Hz, 1H, Hd/Hd′), 7.19 (m, 1H, Hd/Hd′), 7.14 (m, 2H, Hb+Hb′), 4.40 (s, 1H, H2/H4), 4.31 (s, 1H, H2/H4), 3.82 (s, 1H, H9), 2.42 (AB system, δ_{A}=2.92, δ_{B}=1.93, J_{AB}=12.3 HZ, V_{A}=1167 Hz, V_{B}=774 Hz, 2H, H6/H8), 2.36 (t, J=6.9 Hz, 2H, H14), 2.29 (AB system, δ_{A}=2.75, δ_{B}=1.83, J_{AB}=11.2 Hz, V_{A}=1102 Hz, V_{B}=731 Hz, 2H, H6/H8), 2.20 (m, 1H, H10), 1.51 (s, 3H, CH₃), 1.47-1.36 (m, 2H, H11), 1.20 (m, 2H, H13), 1.10 (m, 2H, H12).

¹³C-NMR (100 MHZ, D₂O): δ 181.4 (COOH), 179.1 (COOH), 178.6 (COOH), 159.9 (Cpy/Cpy′), 159.3 (Cpy/Cpy′), 149.2 (2C, Ca+Ca′), 137.0 (2C, Cc+Cc′), 126.0 (Cd/Cd′), 125.5 (Cd/Cd′), 122.9 (Cb/Cb′), 122.8 (Cb/Cb′), 75.3 (C10), 74.3 (C9), 67.8 (C2/C4), 67.1 (C2/C4), 55.9 (C6/C8), 53.3 (C5/C8), 51.7 (C1/C5), 51.5 (C1/C5), 42.8 (CH₃), 40.2 (C14), 32.0 (C13), 29.6 (C11), 23.0 (C12).

IR (cm⁻¹, ATR) v 3342 (broad, O—H alcool/N—H amine), 2949 (broad, O—H acid), 1584, 1568 (s, C═C aromatic/N—H amine), 1366 (s, C—O).

Electrospray ionization ESI/MS⁺: m/z=528.25 ([M+H]⁺, 100%), 529.25 ([M+H]⁺, 28.1%), 530.25 ([M+H]⁺, 3.8%).

Elemental analysis calculated for C₂₆H₃₁ON₅Na2.3H₂O: C, 49.91, H, 5.96, N, 11.19. Found: C, 49.79, H, 5.92, N, 11.19.

### Example 2: Synthesis of Compound (I-a)

Compound (I-a) was prepared according to the following scheme 3:

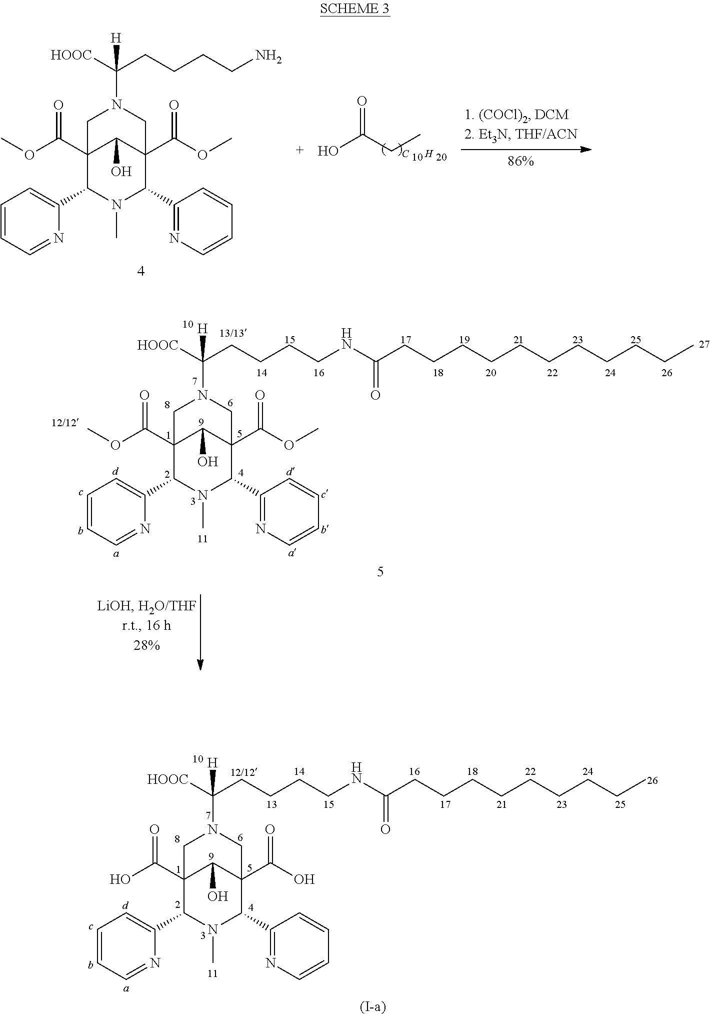

Compound 5. Oxalyl chloride (58 μl, 0.68 mmol, 3 eq) is dissolved in a solution of dodecanoic acid (67 mg, 0.33 mmol, 1.5 eq) at 0° C. under argon. The mixture is stirred at room temperature for 3 hours. Then, the solvent is distilled at the vacuum ramp fitted with a trap and the colourless oil is dissolved in a mixture of ACN (acetonitrile) (4 ml) and THF (4 ml) to form a mixture. Compound 4 (150 mg, 0.22 mmol, 1 eq) and Et₃N (94 μl, 0.67 mmol, 3 eq) are added to said mixture and the resulting solution is stirred at room temperature for 16 hours. The solvent is then removed under vacuum and the crude product is purified by FPLC (C₁₈, 97/3 H₂O/ACN to 50/50 H₂O/ACN) to give 5 (140 mg) with a 86% yield.

¹H NMR (MeOD, 400 MHz): δ 0.90 (t, J=6.9 Hz, 3H, H27); 1.29 (m, 16H, H19-26); 1.63 (m, 4H, H14/H15+H18); 1.74 (m, 2H, H14/H15); 1.89 (m, 1H, H13/13′); 2.21 (t, J=7.4 Hz, 2H, H17); 2.31 (m, 4H, H13/13′+H11); 3.13 (m, 3H, H6/H8); 3.29 (m, 2H, H16); 3.39 (dd, J₁=10.0 Hz, J₂=4.5 Hz, 1H, H10); 3.57 (m, 1H, H6/8); 3.70 (s, 3H, H12/12′); 3.74 (s, 3H, H12/12′); 4.41 (s, 1H, H9); 5.63 (s, 1H, H2/4); 5.64 (s, 1H, H2/4); 7.53 (m, 2H, Hb/b′); 7.77 (m, 2H, Hd/d′); 7.95 (td, J₁=7.8 Hz, J₂=1.8 Hz, 2H, Hc/c′); 8.79 (m, 2H, Ha/a′).

¹³C NMR (MeOD, 126 MHZ): δ 13.08; 22.34; 24.03; 25.77; 28.56; 28.95; 29.07 (2 CH₂); 29.22; 29.27; 29.34 (2 CH₂); 31.76; 35.84; 38.88; 41.92; 48.87; 51.84; 51.88; 51.95; 52.32; 54.63; 65.74; 66.28; 66.93; 72.58; 124.71; 124.76; 127.28; 127.61; 137.51; 137.63; 149.11; 149.64; 151.51; 151.63; 166.94; 169.08; 172.32; 174.96.

Mass Spectrometry: (ESI+/MS): m/z=738.43 [M+H]⁺, calculated for C₄₀H₅₉N₅O₈: 737.44.

Compound (I-a). Lithium hydroxide (23 mg, 0.95 mmol, 5.3 eq) is dissolved in a solution of compound 5 (140 mg, 0.18 mmol, 1 eq) in THF (6 ml) and H₂O (14 ml). The mixture is stirred at room temperature for 2 days. At the end, the solvent is removed under vacuum, the crude product is dissolved in a minimum of water and the pH is lowered to 2. The solution is purified by FPLC (C₁₈, 97/3 H₂O/ACN to 100% ACN) to give compound (I-a).2.5LiCl (48 mg) with a 28% yield.

¹H NMR (MeOD, 400 MHZ): δ 0.9 (t, J=7.2 Hz, 3H, H26); 1.29 (m, 16H, H18-25); 1.63 (m, 4H, H13/H14+H17); 1.74 (m, 2H, H13/H14); 1.89 (m, 1H, H12/12′); 2.21 (m, 2H, H16); 2.31 (m, 4H, H12/12′+H11); 3.11 (m, 3H, H6/H8); 3.30 (m, 2H, H15); 3.36 (m, 1H, H10); 3.57 (m, 1H, H6/8); 4.43 (s, 1H, H9); 5.58 (s, 1H, H2/4); 5.59 (s, 1H, H2/4); 7.53 (m, 2H, Hb); 7.77 (m, 2H, Hd); 7.95 (td, J₁=7.8 Hz, J₂=1.8 Hz, 2H, Hc); 8.79 (m, 2H, Ha).

Mass Spectrometry: (ESI+/HRMS): m/z=710.4113 [M+H]⁺, calculated for C₃₈H₅₅N₅O₈: 709.41.

Elemental Analysis: Calculated for C₃₈H₅₅N₅O₈.2.5 (LiCl), C, 55.94, H, 6.79, N, 8.58. Found: C,56.38, H, 6.71, N, 8.17.

### Example 3: Synthesis of Compound (I-b)

Compound (I-b) was prepared according to the following scheme 4:

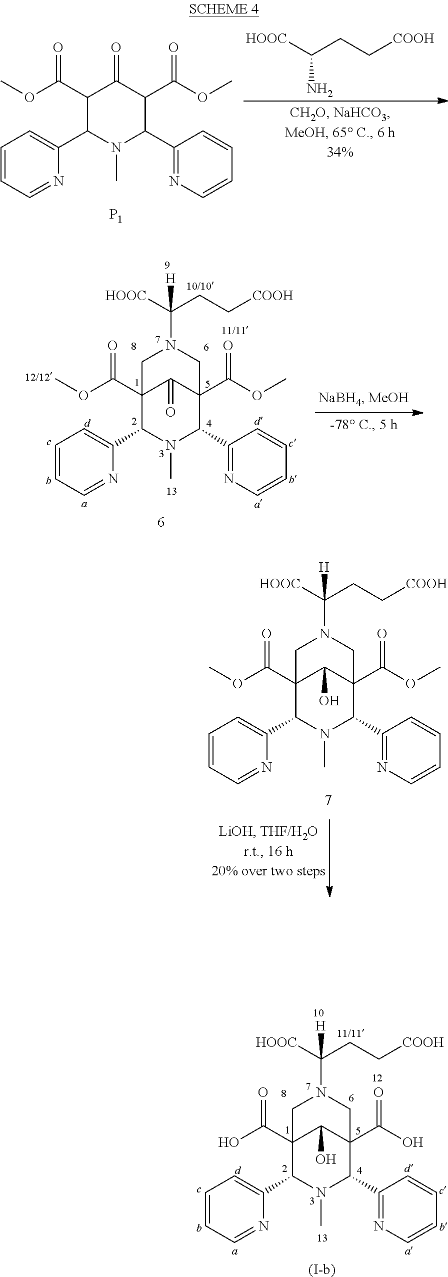

Bispidone 6. L-glutamic acid (422 mg, 2.87 mmol, 1.1 eq), NaHCO₃ (241.5 mg, 2.87 mmol, 1.1 eq) and formaldehyde 37% (635 μl, 8.52 mmol, 3.3 eq) are added to a solution of P1 (1 g, 2.61 mmol, 1 eq) in MeOH and the mixture is stirred at reflux for 2 days. At the end of the reaction, the solvent is removed under vacuum and the crude product is precipited in CH₃CN to give bispidone 6 (500 mg) as a white powder with a 34% yield.

¹H NMR (MeOD, 400 MHz): δ 1.86 (m, 4H, H13+H10/10′); 2.22 ppm (m, 1H, H10/10′); 2.24 (m, 1H, H11/11′); 2.37 (m, 1H, H11/11′); 2.56 (m, 2H, H6/8+H9); 2.77 (m, 1H, H6/8); 3.40 (m, 1H, H6/8); 3.58 (m, 1H, H6/8); 3.69 (s, 3H, H12/12′); 3.76 (s, 3H, H12/12′); 4.65 (s, 1H, H2/4); 4.74 (s, 1H, H2/4); 7.34 (m, 2H, Hb/b′); 7.41 (m, 1H, Hd/d′); 7.44 (m, 1H, Hd/d′); 7.84 (m, 2H, Hc/c′); 8.84 (m, 2H, Ha/a′).

¹³C NMR (MeOD, 126 MHZ): δ 27.02 (C10); 33.87 (C11); 41.84 (C₁₃); 51.66 (C12/12′); 51.86 (C12/12′); 55.62; (C6/8); 57.37 (C6/8); 62.75 (C4); 62.82 (C4); 72.24 (C2/4); 72.50 (C2/4); 73.92 (C9); 123.78 (Cb/b′); 124.10 (Cb/b′); 124.74 (Cd/d′); 124.86 (Cd/d′); 137.60 (Cc/c′); 137.83 (Cc/c′); 150.40 (Ca/a′); 150.58 (Ca/a′); 155.96 (C4); 156.21 (C4); 167.34 (C4); 167.70 (C4); 177.59 (C4); 179.36 (C4); 202.15 (C4).

Compound (I-b). In a solution of bispidone 6 (500 mg, 0.90 mmol, 1 eq) in MeOH at −78° C., sodium borohydride (32 mg, 0.85 mmol, 0.94 eq in total) is added three times (0.25 eq, 0.25 eq and then 0.5 eq the third time) at 1 hour of interval and the mixture is stirred at −78° C. for 2 hours. After two hours, the reaction is quenched with NH₄Cl (5 ml) and the solvent is removed under vacuum. Then, DCM is added and the salts are filtered. The solvent is evaporated and the crude product is purified by reverse phase FPLC (C₁₈, CH₃CN/H₂O). The desired product can be identified in the NMR spectra but others species that were attributed to a partial hydrolysis of the methylester groups are present so the mixture is engaged in the deprotection step without any further purification. The isolated product (200 mg) is thus dissolved in a mixture of THF (3 ml) and H₂O (7 ml). Lithium hydroxide (32 mg, 1.34 mmol) is added and the resulting mixture is stirred at room temperature for 16 hours. The reaction is monitored by TLC on C₁₈ using MeOH/H₂O as eluent. At the end of the reaction, the solvent is removed under vacuum and the crude product is dissolved in a minimun of water to adjust the pH at 2. The solution is finally purified by FPLC (C₁₈, 97/3 H₂O/MeOH to 50/50 H₂O/MeOH) to give compound (I-b).2H₂O (100 mg) with a 20% yield.

¹H NMR (MeOD, 400 MHZ): δ 2.11 (m, 1H, H11/11′); 2.21 (s, 3H, H13); 2.41 (m, 1H, H11/11′); 2.79 (m, 2H, H12); 2.95 (m, 1H, H6/8); 3.10 (m, 2H, H6/8); 3.36 (m, 1H, H6/8); 3.48 (m, 1H, H10); 4.3 (s, 1H, H9); 5.29 (s, 2H, H2/4); 7.57 (m, 2H, Hb/b′); 7.70 (m, 2H, Hd/d′); 7.99 (m, 2H, Hc/c′); 8.75 (m, 2H, Ha/a′).

¹³C NMR (D₂O, 150 MHZ): δ 24.53; 31.08; 41.73; 50.34; 51.05; 56.08; 63.03; 67.44; 67.68; 68.06; 73.65; 123.60 (2CH); 126.03; 126.28; 137.06 (2CH); 148.74; 149.11; 154.39; 154.58; 171.96; 173.39; 173.89; 175.83.

Mass Spectrometry: (ESI+/MS): m/z=529.19 [M+H]⁺, calculated for C₂₅H₂₈N₄O₉: 528.19.

Elemental Analysis: Calculated for C₂₅H₂₈N₄O₉.2 (H₂O), C, 53.19, H, 5.71, N, 9.92. Found: C, 53.07, H, 5.59, N, 10.06.

### Example 4: Synthesis of Compound (I-c)

Compound (I-c) was prepared according to the following scheme 5:

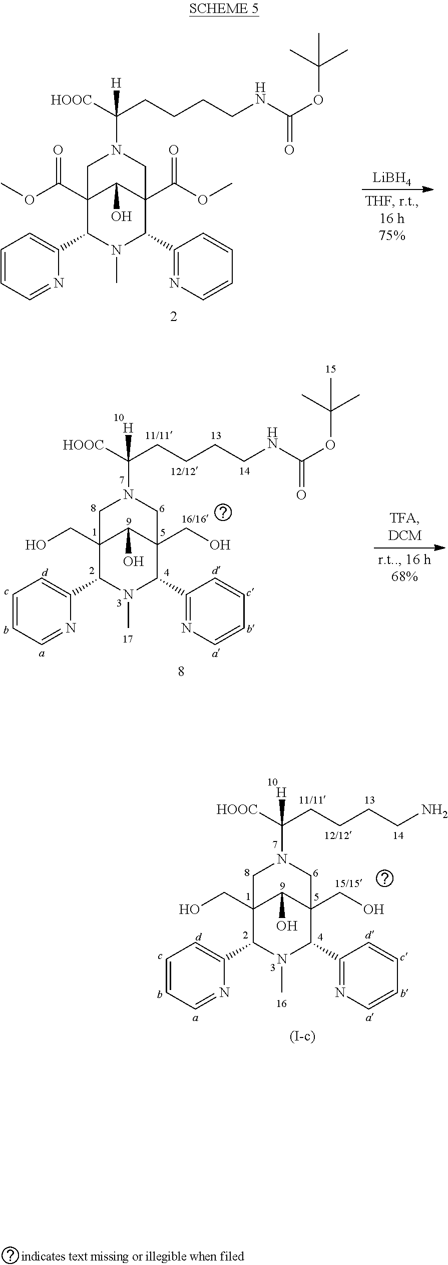

Bispidol 8. In a solution of bispidol 2 (250 mg, 0.38 mmol, 1 eq) in 20 ml of THF and 2 ml of MeOH, 3 eq of LiBH₄ (82 mg, 3.76 mmol, 10 eq) are added in two times at 1 hour of interval and the mixture is stirred during 3 hours at room temperature. The reaction is monitored by TLC on C₁₈ using H₂O/MeOH (90/10) as eluent. At the end, the reaction is quenched with NH₄Cl_{(aq)} (5 ml) and the solvents are removed under vacuum. DCM is added and the salts are filtered. The solvent is evaporated, and the crude product is purified by FPLC (C₁₈, 95/5 H₂O/ACN, to 100% ACN) to give 8 (170 mg) with a 75% yield.

¹H NMR (MeOD, 400 MHZ): δ 1.42 (m, 15H, H15+H11/11′+H12/12′+H13); 1.72 (s, 3H, H17); 2.13 (AB system, δ_{A}=1.67, δ_{B}=2.6, J_{AB}=11.3 Hz, 2H, H6/8ₐₓ+H6/8_{eq}); 2.29 (dd, J₁=10.2 Hz, J₂=4.8 Hz, 1H, H10); 2,31 (AB system, δ_{A}=1.82, δ_{B}=2.79, J_{AB}=11.3 Hz, 2H, H6/8ₐₓ+H6/8_{eq}); 3.00 (m, 2H, H14); 3.16 (AB system, δ_{A}=2.86, δ_{B}=3.45, J_{AB}=11.0 Hz, 2H, H16/16′); 3.32 (AB system, δ_{A}=3.09, δ_{B}=3.56, J_{AB}=11.0 Hz, 2H, H16/16′); 4.07 (s, 1H, H2/4); 4.12 (s, 1H, H9); 4.13 (s, 1H, H2/4); 7.36 (m, 4H, Hb/b′+Hd/d′); 7.74 (m, 2H, Hc/c′); 8.71 (m, 2H, Ha/a′).

¹³C NMR (MeOD, 126 MHZ): δ 23.37 (CH₂lys); 27.48 (C15); 29.7 (CH₂-lys); 29.95 (CH₂-lys); 39.84 (C14); 41.93 (C4); 42.56 (C17+C4); 52.03 (C6/8); 54.99 (C6/8); 65.02 (C16/16′); 65.50 (C16/16′); 67.42 (C2/4); 68.15 (C2/4); 72.68 (C9); 75.23 (C10); 78.42 (C4); 122.59 (Cb/b′); 122.95 (Cb/b′); 125.24 (Cd/d′); 125.70 ((Cd/d′); 136.44 (Cc/c′); 136.60 (Cc/c′); 149.81 (Ca/a′); 150.03 (Ca/a′); 157.10 (C4); 158.98 (C4); 159.63 (C4); 179.55 (C4).

Mass Spectrometry: (ESI+/MS): m/z=600.34 [M+H]⁺, calculated for C₃₁H₄₅N₅O₇: 599.33.

Compound (I-c). TFA (2 ml) is added to a solution of bispidol 8 (170 mg, 0.28 mmol, 1 eq) in 7 ml of DCM. The mixture is stirred for 16 hours at room temperature. At the end of the reaction, the solvent is removed under vacuum and the crude product is purified by FPLC (C₁₈, 97/3 H₂O/ACN, 0.1% TFA to 100% ACN, 0.1% TFA) to give compound (I-c).3TFA (160 mg) with a 68% yield. ¹H NMR (MeOD, 400 MHZ): § 1.60 (m, 2H H11/11′); 1.90 (m, 3H, H12/12′+H13); 2.29 (m, 4H, H16+H12/12′); 2.68 (AB system, δ_{A}=2.59, δ_{B}=2.77, J_{AB}=11.7 Hz, 2H, H6/8ₐₓ+H6/8_{eq}); 2.80 (AB system, δ_{A}=2.53, δ_{B}=3.07, J_{AB}=11.7 Hz, 2H, H6/8ₐₓ+H6/8_{eq}); 3.03 (m, 2H, H14); 3.34 (AB system, δ_{A}=3.12, δ_{B}=3.55, J_{AB}=11.0 Hz, 2H, H15/15′+H15/15′); 3.37 (AB system, δ_{A}=3.19, δ_{B}=3.55, J_{AB}=11.0 Hz, 2H, H15/15′+H15/15′); 3.37 (dd, J₁=9.5 Hz, J₂=4.5 Hz, 1H, H10); 4.29 (s, 1H, H9); 5.12 (s, 1H, H2/4); 5.18 (s, 1H, H2/4); 7.56 (m, 4H, Hd/d′+Hb/b′); 7.94 (m, 2H, Hc/c′); 8.80 (m, 2H, Ha/a′).

¹³C NMR (MeOD, 126 MHZ): δ 23.73 (CH₂lys); 27.42 (CH₂lys); 28.47 (CH₂lys); 39.32 (C14); 41.29 (C17); 42.74 (C4); 43.11 (C4); 48.60 (C6/8); 54.39 (C6/8); 63.57 (C15/15′); 63.70 (C15/15′); 66.69 (C10); 67.71 (C2/4); 68.18 (C2/4); 71.70 (C9); 124.47 (Cb/b′); 124.53 (Cb/b′); 125.52 (Cd/d′); 125.65 (Cd/d′); 137.57 (Cc/c′); 149.47 (Ca/a′); 150.00 (Ca/a′); 151.60 (C4); 172.36 (C4).

Mass Spectrometry: (ESI+/MS): m/z=500.29 [M+H]⁺, calculated for C₂₆H₃₇N₅O₅: 499.28.

Elemental Analysis: Calculated for C₂₆H₃₇N₅O₅.3TFA: C, 45.80, H, 4.94, N, 8.48. Found: C, 45.66, H, 4.79, N, 8.32.

### Example 5: Synthesis of Compound (I-d)

Compound (I-d) was prepared according to the following scheme 6:

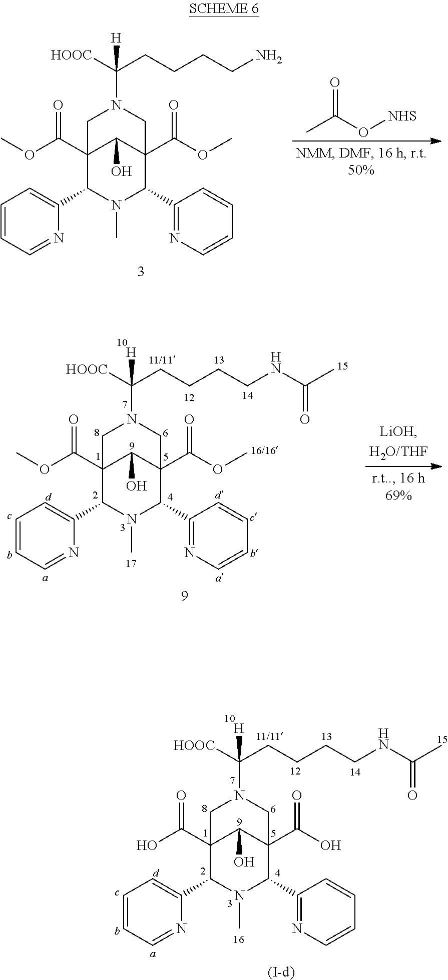

Bispidol 9. N-methylmorpholine (132 μl, 1.2 mmol, 4_{eq}) and N-succinimidyl acetate (59.7 mg, 0.38 mmol, 1.3 eq) are added to a solution of bispidol 3 (200 mg, 0.3 mmol, 1 eq) in 10 ml of DMF. The mixture is stirred for 16 hours at room temperature. At the end, the solvent is removed under vacuum and the crude product is purified by FPLC (C₁₈, 90/10 H₂O/ACN to 100% ACN) to give bispidol 9 (90 mg) with a 50% yield.

¹H NMR (MeOD, 400 MHZ): δ 1.69 (m, 8H, H17+H12+H13); 1.90 (s, 3H, H15); 2.16 (m, 1H, H11/11′); 2.33 (m, 1H, H11/11′); 3.21 (m, 2H, H14); 3.57 (s, 3H, H16/16′); 3.63 (s, 3H, H16/16′); 3.76 (m, 1H, H10); 3.91 (AB system, δ_{A}=3.84, δ_{B}=3.97, J_{AB}=12.2 Hz, 2H, H6/8ₐₓ+H6/8_{eq}); 4.14 (AB system, δ_{A}=4.01, δ_{B}=4.26, J_{AB}=12.2 Hz, 2H, H6/8ₐₓ+H6/8_{eq}); 4.49 (s, 1H, H9); 4.87 (s, 1H, H2/4); 4.92 (s, 1H, H2/4); 7.39 (m, 2H, Hb/b′); 7.60 (d, J=8.0 Hz, 2H, Hd/d′); 7.83 (m, 2H, Hc/c′); 8.65 (m, 2H, Ha/a′); 8.68 (m, 2H, Ha/a′).

¹³C NMR (MeOD, 126 MHZ): δ 22.75 (C15); 25.47 (CH₂-lys); 30.24 (CH₂-lys); 30.42 (CH₂-lys); 40.52 (CH₂-lys); 41.92 (C17); 51.40 (C6/8); 52.54 (C4); 52.59 (C4); 53.15 (C16/16′); 53.25 (C16/16′); 57.01 (C6/8); 67.62 (C2/4); 68.04 (C2/4); 72.12 (C10); 74.04 (C9); 125.11 (Cb/b′); 125.17 (Cb/b′); 128.81 (Cd/d′); 128.88 (Cd/d′); 138.24 (Cc/c′); 138.37 (Cc/c′); 150.35 (Ca/a′); 150.85 (Ca/a′); 157.80 (C4); 157.95 (C4); 170.21 (C4); 170.62 (C4); 173.18 (C4); 173.25 (C4).

Mass Spectrometry: (ESI+/MS): m/z=598.29 [M+H]⁺, calculated for C₃₀H₃₉N₅O₈: 597.28.

Compound (I-d). Lithium hydroxide (32 mg, 35 mmol, 5 eq) is dissolved in a solution of bispidol 9 (160 mg, 0.27 mmol, 1 eq) in a mixture of THF (3 ml) and H₂O (7 ml). The mixture is stirred at room temperature for 16 hours. At the end, the solvent is removed under vacuum. The crude product is dissolved in a minimum of water, the pH of the solution is lowered at 2 and the compound is then purified by FPLC (C₁₈, 97/3 H₂O/ACN to 100% ACN) to give compound (I-d).0.5LiCl (110 mg) with a 69% yield.

¹H NMR (D₂O, 600 MHZ): δ 1.52 (m, 2H, H12); 1.68 (m, 2H, H13); 1.89 (s, 3H, H16); 1.93 (s, 3H, H15); 1.96 (m, 1H, H11/11′); 2.18 (m, 1H, H11/11′);3.35 (AB system δ_{A}=3.08, δ_{B}=3.62, 2H, H6/8ₐₓ+H6/8_{eq}); 3.52 (AB system δ_{A}=3.23, δ_{B}=3.82, 2H, H6/8ₐₓ+H6/8_{eq}); 3.23 (m, 2H, H14); 3.62 (m, 1H, H10); 4.26 (s, 1H, H9); 4.90 (m, 2H, H2/4); 7.42 (m, 2H, Hb); 7.55 (m, 2H, Hd); 7.84 (m, 2H, Hc); 8.59 (m, 2H, Ha).

¹³C NMR (D₂O, 150 MHZ): δ 21.85 (C15); 23.25 (CH₂-lys); 28.06 (CH₂-lys); 28.21 (CH₂-lys); 39.23 (CH₂-lys); 41.90 (C16); 50.19 (C4); 50.72 (C4); 52.07 (C6/8); 54.79 (C6/8) 67.84 (C2/4); 68.00 (C2/4) 71.42 (C10); 72.98 (C9); 123.86 (Cb/b′+Cb/b′); 125.19 (Cd/d′); 126.34 (Cd/d′); 137.59 (Cc/c′); 137.64 (Cc/c′); 148.83 (Ca/a′); 149.09 (Ca/a′); 155.59 (C4); 155.76 (C4); 173.88 (C4); 174.48 (C4); 175.00 (C4); 175.61 (C4).

Mass Spectrometry: (ESI+/MS): m/z=570.25 [M+H]⁺ calculated for C₂₈H₃₅N₅O₈: 569.25.

Elemental Analysis: Calculated for C₂₈H₃₅N₅O₈.0.5 (LiCl), C, 56.92, H, 5.97, N, 11.85. Found: C, 56.82, H, 6.01, N, 11.96.

### Example 6: Synthesis of Compound (I-e)

Compound (I-e) was prepared according to the following scheme 7:

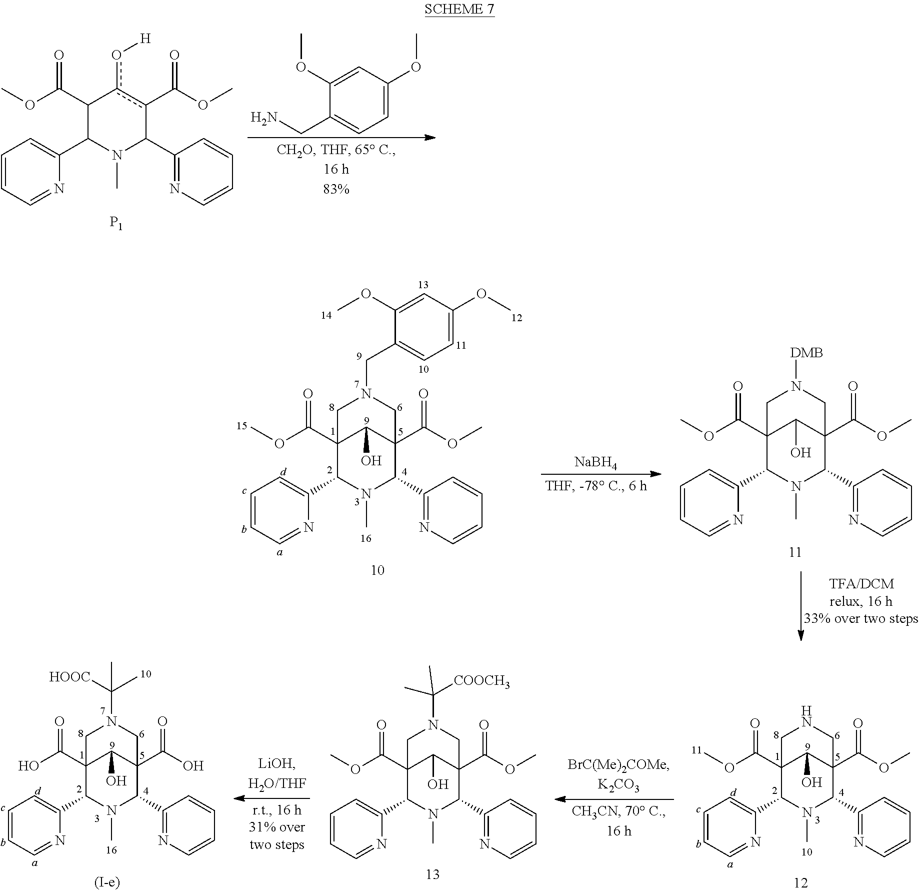

Compound 10. P1 (dimethyl-1-methyl-4-oxo-2,6-di(pyridin-2-yl) piperidine-3,5-dicarboxylate, 1750 mg, 4.56 mmol, 1 eq) is dissolved in THF (40 ml). 2,4-Dimethoxybenzylamine (910 mg, 5.45 mmol, 1.2 eq) and formaldehyde 37% (88 μl, 11 mmol, 2.4_{eq}) are then added successively. The mixture is heated at reflux for 16 hours and the reaction is monitored by TLC (SiO₂, DCM/MeOH as eluent). At the end of the reaction, solvent is removed and the solid is washed with hot MeOH yielding compound 10 as a colourless solid (2.15 g, 83%).

¹H NMR (CDCl₃, 400 MHZ): δ 1.91 (s, 3H, H16); 2.76 (AB system, δ_{A}=2.46; δ_{B}=3.05 d, J_{AB}=12.0 Hz, 4H, H6/8); 3.36 (s, 2H, H9); 3.8 (s, 3H, H12/H14), 3.84 (s, 6H, H15), 3.93 (s, 3H, H12/H14); 4.65 (s, 2H, H2+H4); 6.41 (dd, J₁=8.2 Hz, J₂=2.5 Hz, 1H, H11); 6.57 (d, J=2.5 Hz, 1H, H13); 7.02 (d, J=8.2 Hz, 1H, H10); 7.1 (m, 2H, Hb); 7.46 (td, J₁=7.8 Hz, J₂=1.9 Hz, 2H, Hc); 7.98 (d, J=7.8 Hz, 2H, Hd); 8.34 (m, 2H, Ha).

¹³C NMR (CDCl₃, 126 MHz): δ 43.24; 52.48; 55.56; 55.82; 59.08; 62.11; 74.04; 98.79; 103.79; 117.77; 122.84; 123.74; 133.2; 136.16; 148.90; 158.75; 159.35; 160.77; 168.68; 204.09.

Mass Spectroscopy (ESI+/MS): m/z=575.25 [M+H]⁺, calculated for C₃₁H₃₄N₄O₇: 574.24.

Compound 12. In a solution of compound 10 (6.56 g, 11.4 mmol, 1 eq) in THF at −78° C., 0.33 eq of sodium borohydride (0.28 g, 7.52 mmol, 0.66_{eq} in total) is added two times at 2 hours of interval and the mixture is stirred at −78° C. for 2 hours. At the end, the reaction is quenched with 40 ml of NH₄Cl and the solvent is removed under vacuum. The DCM is added, salts are filtered and the solvent is removed under vacuum. The crude product is used for the next step without purification and it is dissolved in a mixture of DCM (15 ml) and TFA (10 ml). The mixture is stirred at reflux for 16 hours. At the end, the solvent is evaporated and the crude product is dissolved in 20 ml of MeOH and stirred at reflux for 2 hours. A white precipitate is obtained, which is then filtered and the solvent of the solution is removed under vacuum. Finally, the crude product is purified by FPLC (SiO₂, 100% DCM to 80/20 DCM/MeOH) to give compound 12 (1.62 g) with a 33% yield.

¹H NMR (MeOD, 400 MHZ): δ 1.53 (s, 3H, H10); 3.60 (s, 6H, H11); 3.96 (AB system, δ_{A}=3.60, δ_{B}=4.31, J_{AB}=13.4 Hz, 4H, H6/8ₐₓ+H6/8_{eq}); 4.53 (s, 1H, H9); 4.75 (s, 2H, H2/4); 7.35 (m, 2H, Hb/b′); 7.54 (d, J=7.6 Hz, 2H, Hd/d′); 7.97 (td, J₁=7.6 Hz, J₂=1.9 Hz, 2H, Hc/c′); 8.64 (m, 2H, Ha/a′).

Compound (1-e). Potassium carbonate (106.4 mg, 0.77 mmol, 1.1 eq) is dissolved in a solution of compound 12 (300 mg, 0.70 mmol, 1 eq) in 30 ml of anhydrous ACN under argon. Then, methyl 2-bromo-2-methylpropionoate (100 μl, 0.77 mmol, 1.1_{eq}) is added and the mixture is heated at 80° C. for 16 hours. At the end, K₂CO₃ is filtered and solvent is removed under vacuum. The product is used for the next step without purification. The crude product is dissolved in a mixture of H₂O (20 ml) and THF (10 ml) and lithium hydroxide (83.82 mg, 3.5 mmol, 5 eq) is added. The solution is stirred for 16 hours at room temperature. At the end, the solvent is evaporated and the crude product is dissolved in a minimum of H₂O and the pH is lowered to 2 with aqueous HCl. The solution is then purified by FPLC (C₁₈, 97/3 H₂O/MeOH to 50/50 H₂O/MeOH) to give compound (1-e).2HCl.0.5H₂O (125 mg) with a 31% yield.

¹H NMR (MeOD, 400 MHZ): δ 1.13 (s, 6H, H11); 2.08 (s, 3H, H10); 2.50 (AB system, δ_{A}=2.39, δ_{B}=2.60, J_{AB}=12.8 Hz, 4H, H6/8ₐₓ+H6/8_{eq}); 4.11 (s, 1H, H9); 5.03 (s, 2H, H2+H4); 7.66 (m, 4H, Hb+Hd); 8.06 (td, J₁=7.9 Hz, J₂=1.3 Hz, 2H, Hc); 8.73 (m, 2H, Ha).

¹³C NMR (MeOD, 126 MHZ): δ 23.15; 46.61; 51.41; 53.23; 53.79; 67.70; 68.74; 75.18; 128.14; 128.93; 142.13; 150.87; 157.81; 178.60; 186.65.

Mass Spectrometry: (ESI+/MS): m/z=485.19 [M+H]⁺, calculated for

C₂₄H₂₈N₄O₇: 484.20.

Elemental Analysis: Calculated for C₂₄H₂₈N₄O₇.2HCl.0.5H₂O, C, 50.89, H, 5.52, N, 9.89. Found: C, 50.77, H, 5.71, N, 10.33.

### Example 7: Synthesis of Compound (I-f)

Compound (I-f) was prepared according to the following scheme 8:

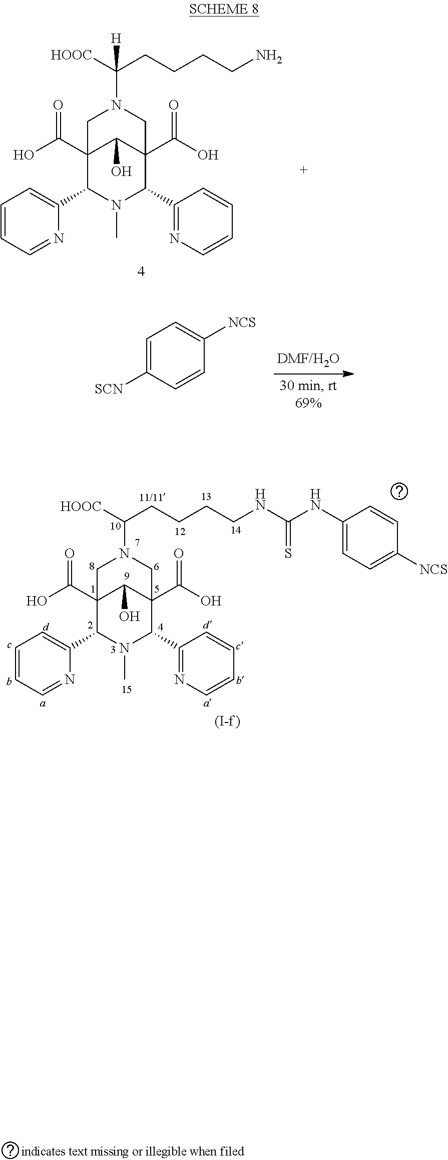

Compound (I-f). Compound 4 (70 mg, 0.13 mmol, 1 eq) is dissolved in H₂O (2.5 ml) and DMF (2 ml). p-phenylene diisothiocyanate (PDITC) (291 mg, 1.33 mmol, 10 eq) is then added to the solution and the resulting mixture is stirred during 30 min. At the end, the excess of PDITC is filtered. H₂O is added to the solution and the observed precipitate is filtered. The solvent is then removed under vacuum at 35° C. to finally give compound (I-f). 3NaOH. H₂O (77 mg) with a 69% yield.

¹H NMR (D₂O, 400 MHZ): δ 1.62 (m, 2H, H12); 1.79 (m, 2H, H13); 1.88 (s, 3H, H15); 2.00 (m, 1H, H11/H11′); 2.20 (m, 1H, H11/H11′); 3.17 (AB system, δ_{A}=3.13, δ_{B}=3.21, J_{AB}=12.3 Hz, 2H, H6/H8); 3.56 (m, 4H, H10+H14+H8/H6); 3.81 (m, 1H, H6/H8); 4.28 (s, 1H, H9); 4.91 (m, 2H, H2+H4); 7.23 (m, 2H, He/Hf), 7.31 (m, 2H, He/Hf); 7.43 (m, 2H, Hd/Hd′); 7.59 (m, 2H, Hb/Hb′); 7.86 (m, 2H, Hc/Hc′); 8.64 (m, 2H, Ha/Ha′).

Mass Spectrometry: (ESI+/MS): m/z=720.23 [M+H⁺], calculated for C₃₄H₃₇N₇O₇S₂, 729.22.

Elemental Analysis: Calculated for C₃₆H₄₁N₇O₇S₂.3NaOH·H₂O, C, 47.60, H, 4.93, N, 11.43, S, 7.48. Found: C, 47.89, H, 4.92, N, 11.61, S, 7.24.

### Example 8: Synthesis of Compound (I-g)

Compound (I-g) was prepared according to the following scheme 9:

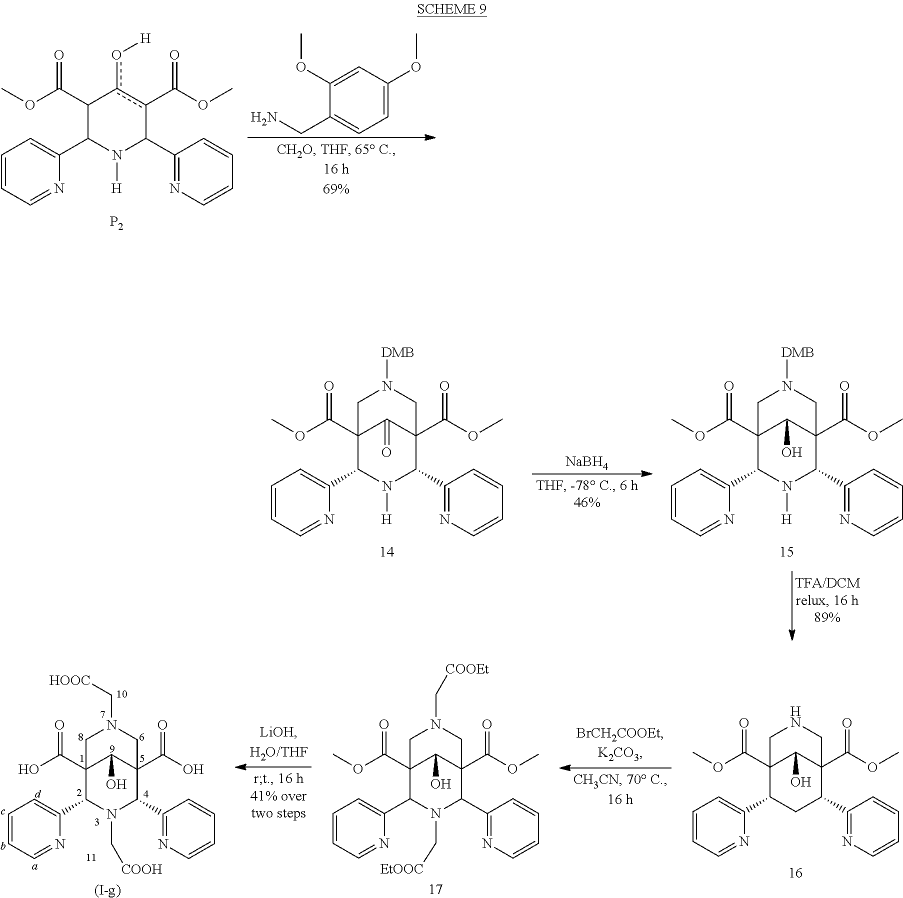

P₂ is a well-known starting material to lead to bispidone and bispidine compounds.

Compound 14. Piperidone P2 (20.0 g, 54.2 mmol, 1.0_{eq}) was dissolved in 200 ml tetrahydrofuran and heated to 50° C. 2,4-dimethoxybenzylamine (49.8 ml, 65.0 mmol, 1.2 eq) and formaldehyde (9.76 ml, 37% in MeOH/H₂O, 130 mmol, 2.4 eq) were added successively via a dropping funnel. The reaction mixture was heated under reflux overnight. After cooling to room temperature the solvent was removed under reduced pressure and the remaining solid was washed with hot MeOH yielding compound 14 as a colorless solid (20.8 g, 37.0 mmol, 69%). ¹H NMR (600.13 MHZ, 25° C., CDCl₃): δ=2.92 (d, ²J_{H,H}=11.5 Hz, 2 H, N7CH_{2ax,eq}), 3.28 (bs, 2 H, N7CH₂), 3.61-3.62 (m, 5H, N7CH_{2ax,eq}, o-OCH₃), 3.74 (s, 6H, COOCH₃), 3.79 (s, 3H, p-OCH₃), 5.10 (bd, ³J_{H,H}=11.6 Hz, 2 H, N3CH), 5.22 (bt, ³J_{H,H}=11.6 Hz, 1H, N3H), 6.27 (bd, ³J_{H,H}=7.3 Hz, 1H, Hph), 6.30 (d, ⁴J_{H,H}=1.9 Hz, 1H, Hph), 6.96 (d, ³J_{H,H}=7.3 Hz, 1H, Hph), 7.13 (bt, ³ J_{H,H}=4.2 Hz, 2 H, Hpy), 7.53 (d, ³J_{H,H}=7.0 Hz, 2 H, Hpy), 7.61 (bt, ³J_{H,H}=7.0 Hz, 2 H, Hpy), 8.37 (bd, ³J_{H,H}=4.2 Hz, 2 H, Hpy) ppm.

¹³C NMR (150.92 MHZ, 25° C., CDCl₃): δ=52.1, 54.9, 55.4, 55.4, 57.6, 61.3, 66.5, 98.3, 103.3, 118.0, 122.3, 122.8, 131.7, 136.2, 148.5, 157.5, 158.9, 160.2, 169.9, 205.5 ppm.

Compound 15. Compound 14 (6.00 g, 10.7 mmol, 1.0_{eq}) was dissolved in THF and cooled to −78° C. Sodium borohydride (203 mg, 5.35 mmol, 0.5_{eq}) was added in two times at 2 hours of interval and the solution was stirred overnight at −78° C. for 2 hours. At the end, the reaction is quenched with NH₄Cl and the solvent is removed under vacuum. The DCM is added, salts are filtered and the solvent is removed under vacuum. The crude product is used for the next step without purification (46% yield).

Compound 16. Compound 15 is dissolved in a mixture of DCM (15 ml) and TFA (10 ml). The mixture is stirred at reflux for 16 hours. At the end, the solvent is evaporated and the crude product is dissolved in 20 ml of MeOH and stirred at reflux for 2 hours. A white precipitate is obtained, which is then filtered and the solvent of the solution is removed under vacuum. The TFA salt of compound 16 was obtained as colorless crystals (89% yield) from the concentrated filtrate.

¹H NMR (600.13 MHZ, 25° C., MeOH-d⁴): δ=3.48 (dd, ²J_{H,H}=13.2 Hz, ³ J_{H,H}=1.2 Hz, 2 H, N7CH_{2ax,eq}), 3.62 (bd, ²J_{H,H}=13.2 Hz, 2 H, N7CH_{2ax,eq}), 3.69 (s, 6H, COOCH₃), 4.76 (d, ³J_{H,H}=1.4 Hz, 2 H, N3CH), 4.89 (s, 1H, CHOH), 7.40 (ddd, ³J_{H,H}=7.7 Hz, ³J_{H,H}=4.8 Hz, ⁴J_{H,H}=1.2 Hz, 2 H, Hpy), 7.49 (d, 3J_{H,H}=7.7 Hz, 2 H, Hpy), 7.85 (td, ³J_{H,H}=7.7 Hz, ⁴J_{H,H}=1.8 Hz, 2 H, Hpy), 8.63 (ddd, ³J_{H,H}=4.8 Hz, ³J_{H,H}=1.8 Hz, ⁴J_{H,H}=0.9 Hz, 2 H, Hpy) ppm.

¹³C NMR (150.92 MHZ, 25° C., MeOH-d⁴): δ=41.4, 51.9, 53.2, 67.6, 73.3, 124.7, 125.3, 139.0, 150.8, 158.2, 171.6 ppm.

HR-ESI MS (pos, MeOH): [5+H]⁺ calcd 413.18195, obsd 413.18221.

Elemental analysis (report no. 39141): [5.H (TFA)]calcd C, 52.47; H, 4.79; N, 10.64%; obsd C, 52.72; H, 4.93; N, 10.57%.

Compound (I-g). Potassium carbonate (212 mg, 1.53 mmol, 2.4_{eq}) is dissolved in a solution of compound 16 (263 mg, 0.64 mmol, 1_{eq}) in 50 ml of anhydrous ACN under argon. Then, ethyl bromoacetate (182 μl, 1.6 mmol, 2.5 eq) is added and the mixture is heated at 80° C. for 16 hours. At the end, K₂CO₃ is filtered and the solvent is removed under vacuum. The crude product containing compound 17 is used for the next step without purification. The crude product is dissolved in a mixture of H₂O (10 ml) and THF (5 ml) and lithium hydroxide (123 mg, 5.12 mmol, 8 eq). The solution is stirred for 16 hours at room temperature. At the end, the solvent is evaporated and the crude product is purified by FPLC (C₁₈, 97/3 H₂O/MeOH to 50/50 H₂O/MeOH) to give compound (I-g). 2 LiOH. H₂O (150 mg) with a 41% yield.

¹H NMR (MeOD, 400 MHZ): δ 2.82 (s, 2H, H10/H11); 3.68 (AB system, δ_{A}=3.60, δ_{B}=3.75, 4H, H6/8ₐₓ+H6/8_{eq}); 3.75 (s, 2H, H10/H11); 4.42 (s, 1H, H9); 5.75 (s, 2H, H2/H4); 7.36 (m, 2H, Hb); 7.65 (m, 2H, Hd); 7.80 (m, 2H, Hc); 8.67 (m, 2H, Ha).

¹³C NMR (D₂O, 150 MHZ): δ 50.83; 56.27; 59.10; 64.07; 72.81; 124.66; 127.54; 140.64; 146.92; 155.00; 170.92; 174.71; 176.56.

Mass Spectrometry: (ESI/MS): m/z=499.14 [M−H]; 505.15 [M-2H+Li], calculated for C₂₃H₂₄N₄O₉: 500.15

Elemental Analysis: Calculated for C₂₃H₂₄N₄O₉.2 (LiOH).H₂O, C, 48.78, H, 4.98, N, 9.89. Found: C,48.78, H, 5.02, N, 10.16.

### Example 9: Synthesis of a Complex of Manganese (II) with Bispidine-Based Metal Chelating Ligands (I-a)

A complex (I-A) of manganese (II) with chelating bispidine-based ligand (I-a) of example 2 was prepared as follows:

Equimolar quantities of ligand and MnCl₂ were dissolved in milli-Q water, the pH was adjusted to 7.4 either with a buffered solution or by adding KOH or HCl to the solution and the samples were heated at 60° C. for 30 min. The absence of free Mn²⁺ was checked by the Xylenol orange test.

The properties of complex (I-A) in terms of relaxivity and stability were compared to the properties of two other promising Mn-based constrat agents, namely MnPC2A-EA and MnPyC3A, the latest compound being patented and already tested in clinical trials in baboons.

The same preparation method was used to obtain a complex (I-D) of manganese (II) with chelating bispidine-based ligand (I-d) of example 5.

Table 1 below shows the properties of complexes (I-A) and (I-D), and for comparison of MnPC2A-EA and MnPyC3A.

TABLE 1

| | Longitudinal | |
|---|---|---|
| | relaxivity in water | Half-life in excess of |
| | (r₁; mM⁻¹/s) | Zn²⁺(25 eq, pH = 6, |
| | at 25° C., pH = 7.4, | 37° C.) |
| Compound | and 60 MHz | (t_{1/2}) |
| (I-A) | 4.89 | NA |
| (I-D) | 4.03 | 160 days |
| MnPC2A-EA | 3.52 | 54.4 hours |
| MnPyC3A1 | 3.3 | 0.285 hours |

NA: not available

The relaxivity at 60 MHz is measured with a device commercialized under the brand name WP80 NMR by the firm Brucker according to the following protocol. A millimolar aqueous solution of complex was prepared with Milli-Q water (p<18MΩ). The concentration of Mn²⁺-containing samples was checked by ICP-OES and/or NMR by using the bulk magnetic susceptibility. The absence of free Mn²⁺ was checked by the Xylenol orange test. For the relaxivity measurement, the temperature was monitored by a VTC91 temperature control unit and maintained by a gas flow. The temperature was determined by previous calibration with a Pt resistance temperature probe.

The kinetic inertness of the maganese (II) complexes and in particular their half-life was assessed at 37° C. and in 0.1 M KCl, via transmetallation studies of Mn²⁺ complex (1 mM) with Zn²⁺ (as ZnCl²) at pH 6.06 (0.030 M MES buffer, in the presence of 10- and 50-fold excess of the exchanging Zn²⁺), in the presence of 50-fold excess of Zn²⁺. The excess of the exchanging metal ion guarantees the pseudo-first order conditions. For the duration of the experiments (up to 3 months), the samples were stored in a thermostat at 37° C. between the relaxivity measurements. The pH was controlled for each sample at the end of the kinetic measurements to confirm that it remained stable during the experiment. The reactions were monitored by measuring the water proton relaxation rates at 60 MHz on a relaxometer commercialized under the brand name Minispec by the firm a Bruker. The analysis of the experimental data was performed using Visualiseur/Optimiseur running on a MATLAB 8.3.0 (R2014a) platform according to equations (1) and (2):

$$- \frac{{d [ M n L ]}_{t}}{d t} = {{k}_{o b s} [ M n L ]}_{t} \qquad Equation ( 1 )$$

$${t}_{1 / 2} = ln ( 2 ) / {k}_{o b s}$$

were [MnL]t stands for the total concentration of MnL complex, K_{obs} stands for the pseudo-first order constant and t_{1/2} is the half-life of the MnL complex in the given conditions.

Based on these results in Table 1, it can be concluded that the ligands of the present invention lead to complexes having improved relaxivity while guaranteeing good kinetic inertness. Additionally, the complexes obtained do not involve gadolinium metal and have therefore a reduced toxicity.

### Example 10: Synthesis of Compound (I-h)

Compound (I-h) was prepared according to the following scheme 10:

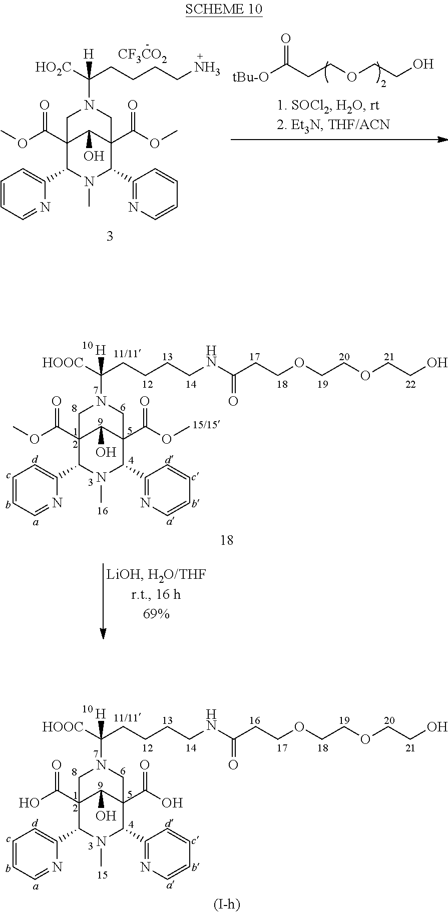

Compound 18. The tBu-ester (31 mg, 0.13 mmol) was dissolved in SOCl₂ (87 μl, 1.19 mmol) in a reaction vial equipped with a magnetic stir bar. Water (2.6 μl, 0.14 mmol) was then added. The reaction mixture was stirred at room temperature over 60 min. Then, toluene (1 ml) was added and the mixture was taken to dryness to give a pale yellow liquid. This intermediate was mixed with a solution of bispidol 3 (71 mg, 0.106 mmol) in ACN (1.8 ml) and THF (1.8 ml). Then, triethylamine (50.0 μl, 0.36 mmol) was added and the reaction mixture was stirred at room temperature for 1.5 hours. The solvents were removed under vacuum and the crude product was purified on normal phase column chromatography (isocratic DCM/MeOH 80/20+0.1% TFA) to give compound 18 (31 mg, 41%).

TLC (SiO₂; DCM/MeOH, 90/10); Rf=0.5.

TLC (C₁₈; ACN/H₂O, 50/50); Rf=0.4.

1H NMR (400 MHZ, CD₃OD): δ 1.56 (m, 2H, H12), 1.65-1.80 (m, 2H, H13), 1.86 (m, 1H, H11/11′), 2.23-2.37 (m, 4H, H16+H11/11′), 2.46 (t, J=6.2 Hz, 2H, H22), 3.16-3.04 (m, 4H, H14+H6/H8), 3.27 (d, J=6.9 Hz, 2H, H6/H8), 3.37 (dd, J₁=10.0 Hz, J₂=4.6 Hz, 1H, H10), 3.53-3.59 (m, 2H, CH₂PEG), 3.62 (bs, 4H, CH₂PEG), 3.63-3.66 (m, 2H, CH₂PEG), 3.68 (s, 3H, H15/15′), 3.72 (s, 3H, H15/15′), 3.75 (t, J=6.2 Hz, 2H, H17), 4.38 (s, 1H, H9), 5.58 (s, 1H, H2/4), 5.60 (s, 1H, H2/4), 7.52 (bt, ³J_{H,H}=7.6 Hz, 2H, Hb+Hb′), 7.89-7.65 (m, 2H, Hd+Hd′), 7.93 (t, ³J_{H,H}=7.7 Hz, 2H, Hc+Hc′), 8.77 (bd, ³J_{H,H}=4.9 Hz, 1H, Ha+Ha′).

Mass Spectrometry: (MALDI-MS): m/z=716.623 [M+H]⁺, calculated for C₃₅H50N5011: 716.351.

Compound (I-h). A solution of lithium hydroxide (0.39 mmol, 9.4 mg) in H₂O (2.8 mL) was added to the solution of compound 17 (0.04 mmol, 28.0 mg) in THF (1.3 mL). The final solution was stirred at room temperature for 30 min. The solvents were evaporated under vacuum and the crude product was solubilized in a minimal volume of water and pH was adjusted below 2 by TFA. The solution was purified by flash column chromatography (C₁₈; H₂O/ACN; 95/5 to 100% ACN) to give the desired compound (I-h) (26 mg, 97%).

HPLC (C₁₈; ACN/H₂O), t_{R}=15.79 min. ¹H NMR (400 MHZ, CD₃OD): § 1.55 (dt, J=15.1, 7.2 Hz, 2H, H12), 1.72 (d, J=7.3 Hz, 2H, H13), 1.85 (dd, J=7.0, 2.9 Hz, 1H, H11/11′), 2.31 (m, 4H, H15+H11/11′), 2.46 (t, J=6.1 Hz, 2H, H21), 2.83-3.17 (m, 3H, H6/H8+H14), 3.28 (m, 1H, H6/H8), 3.34 (1H, H10), 3.49 (dd, J=12.4, 2.2 Hz, 1H, H6/H8), 3.55 (dd, J=5.6, 4.1 Hz, 2H, CH₂PEG), 3.58-3.70 (m, 6H, CH₂PEG), 3.75 (t, J=6.1 Hz, 2H, H17), 4.42 (s, 1H, H9), 5.56 (s, 1H, H2/4), 5.58 (s, 1H, H2/4), 7.34-7.68 (m, 2H, Hb+Hb′), 7.86-7.70 (m, 2H, Hd+Hd′), 8.78 (dt, J=4.8, 1.4 Hz, 2H, Hc+Hc′), 7.86-8.10 (m, 2H, Ha+Ha′).

¹³C NMR (101 MHZ, D₂O): δ 23.48 (CH₂), 26.16 (CH₂), 28.19 (CH₂), 36.04 (CH₂), 38.85 (CH₂), 42.31 (C10), 48.82 (C15), 50.65 (CH₂), 50.94 (CH₂), 51.04 (C1/5), 54.04 (C1/5), 60.32 (CH₂), 65.27 (C2/4), 65.59 (C2/4), 66.79 (CH₂), 69.46 (CH₂), 71.67 (CH₂), 71.99 (C9), 126.05 (CHpy), 126.01 (CHpy), 125.77 (CHpy), 125.73 (CHpy), 127.96 (CHpy x2), 128.02 (CHpy x2), 152.42 (C4), 172.01 (C⁴), 172.12 (C⁴), 173.85 (C⁴x2).

1-15. (canceled)

16. A bispidine-based metal chelating ligand responding to the following formula (I): 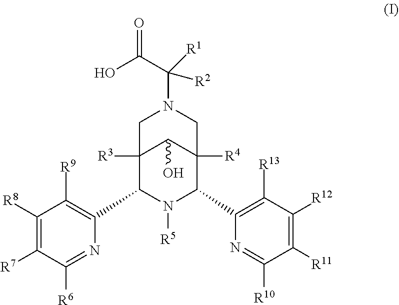
in which:
R¹ represents an hydrogen atom or a C₁-C₈ alkyl group,
R² represents an hydrogen atom, a C₁-C₈ alkyl group, a group responding to formula (II):—(CH₂)ₙ—NH₂ (II), where 3≤n≤18, or a group responding to the following formula (III): 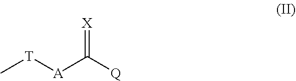
where:
T represents a C₁-C₁₇ alkylene group, a C₁-C₁₇ alkenylene group, or a C₁-C₁₇ alkynylene group,
A represents —CH₂— or —NH—,
X represents an oxygen atom, a sulfur atom, or a NH group, and
Q represents:
an alkyl group optionally substituted with a functional group,
a NHR¹⁴ group where R¹⁴ represents an hydrogen atom, or an aryl group optionally substituted with a functional group,
a polyethylene glycol group responding to formula (IV): —CH₂—(CH₂—O—CH₂)_{q}—CH₂OH(IV), where 1≤q≤24, or
an OH group,
R³ and R⁴, which may be identical or different, represent a CH₂OH group, a CO₂H group, or a CONHR¹⁵ group, where R¹⁵ represents:
an alkyl group optionally substituted with a functional group,
an aryl group optionally substituted with a functional group, or
a polyethylene glycol group responding to formula (V): —CH₂—(CH₂—O—CH₂)ᵣ—CH₂OH(V), where 1≤r≤24,
R⁵ represents an hydrogen atom, an alkyl group, a group of formula (VI): —(CH₂)ₘ—CO₂H(VI) where 1≤m≤5, a polyethylene glycol group responding to formula (VII): —CH₂—(CH₂—O—CH₂)ₛ—CH₂OH(VII), where 1≤s≤18, or a group of formula (III) as defined above,
R⁶, R⁷, R⁸, R⁹, R¹⁰, R¹¹, R¹², and R¹³, which may be identical or different, represent an hydrogen atom, an OH group, an ether group OR¹⁶ where R¹⁶ is an alkyl group, a CO₂H group, or a CONHR¹⁷ group, where R¹⁷ represents an alkyl group optionally substituted with a functional group,
with the proviso that:
when R² represents a group responding to formula (II):—(CH₂)ₙ—NH₂ (II), and R⁵ represents an alkyl group, then R³ and R⁴ are different from CO₂H groups,
when R⁵ represents an alkyl group, then the pair (R¹, R²) is different from the pair (hydrogen atom, hydrogen atom), and
when R² represents a group of said formula (III), where T represents a C₁-C₁₇ alkylene group, A represents —NH—, X represents an oxygen atom, and Q represents a C₁-C₅ alkyl group substituted with a functional group, then R³ and R⁴ are different from CO₂H groups.

17. The ligand according to claim 16, wherein T represents a C₁-C₁₇ alkylene group.

18. The ligand according to claim 16, wherein the alkyl group optionally substituted with a functional group as a Q group is a linear C₅-C₂₀ alkyl group optionally substituted with a functional group.

19. The ligand according to claim 16, wherein the functional group is selected from the following groups: amide, sulfonate, sulfate, quaternary ammonium, hydroxyl, phosphonate, succinimidyl ester, sulfosuccinimidyl ester, isothiocyanate, isocyanate, iodoacetamide, maleimide, sulfonyl halide, acid halide, carbodiimide, biotin, azido, alkyne, and a —C(O)—Z group, where Z is selected from an —OH, —N-glycine, —N-lysine, —Y-L-NH₂, —Y-L-COOH and —Y-L-SH group, where Y is selected from N and O atoms, and L is selected from a C₁-C₁₇ alkylene group, a C₁-C₁₇ alkenylene group, a C₁-C₁₇ alkynylene group, and a phenylene group.

20. The ligand according to claim 16, wherein the NHR¹⁴ group as the Q group is such that R¹⁴ represents a C₅-C₂₀ aryl group optionally substituted with a functional group.

21. The ligand according to claim 16, wherein R² represents a group responding to said formula (II) or (III).

22. The ligand according to claim 16, wherein the group of formula (III) is such that:
A represents —NH—, X represents an oxygen atom, and Q represents an alkyl group optionally substituted with a functional group or a polyethylene glycol group responding said formula (IV);
A represents —NH—, X represents a sulfur atom, and Q represents a NHR¹⁴ group where R¹⁴ represents an aryl group optionally substituted with a functional group; or
A represents —CH₂—, X represents an oxygen atom, and Q represents an OH group.

23. The ligand according to claim 16, wherein R³ and R⁴, which may be identical or different, represent a CH₂OH group or a CO₂H group.

24. The ligand according to claim 16, wherein R³ and R⁴ are identical.

25. The ligand according to claim 16, wherein R⁵ represents an alkyl group or a group of formula (VI):—(CH₂)ₘ—CO₂H(VI) where 1≤m≤5.

26. The ligand according to claim 16, wherein the alkyl group as R⁵ is a linear C₁-C₅ alkyl group.

27. The ligand according to claim 16, wherein R⁶, R⁷, R⁸, R⁹, R¹⁰, R¹¹, R¹², and R¹³ groups represent hydrogen atoms or at least R⁷, R⁹, R¹¹, and R¹³ represent hydrogen atoms.

28. The ligand according to claim 16, wherein it is selected from the following formulae (I-a) to (I-h):

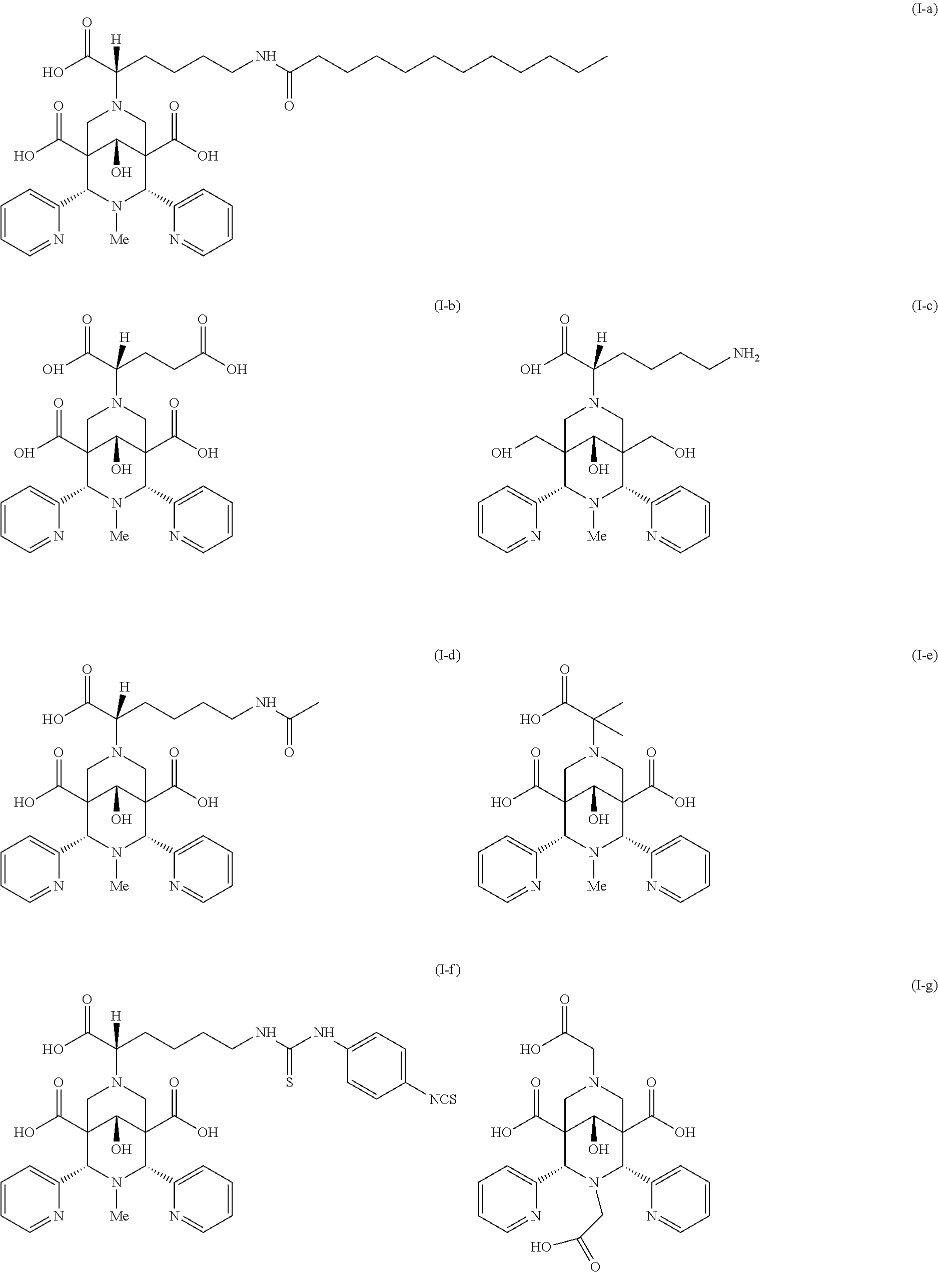

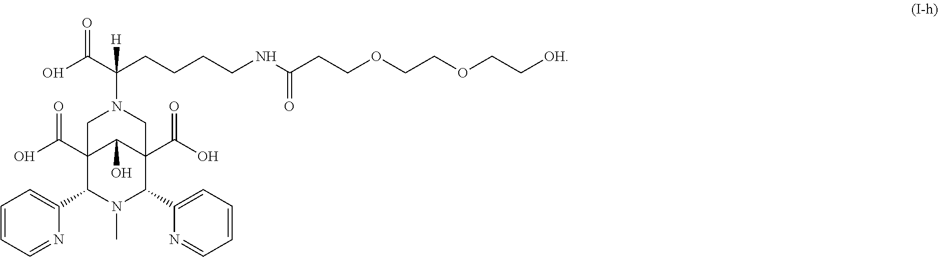 [SMILES: CN1[C@@H](c2ccccn2)C2(C(=O)O)CN([C@@H](CCCCNC(=O)CCOCCOCCO)C(=O)O)CC(C(=O)O)([C@H]2O)[C@H]1c1ccccn1]

29. A complex of a metal M, wherein said complex comprises a metal ion of said metal M complexed with a bispidine-based metal chelating ligand (I) according to claim 16, said metal M being selected from copper, manganese, gallium, cobalt, zinc, nickel, and iron.

30. A magnetic resonance imaging contrast agent, comprising a bispidine-based metal chelating ligand (I) according to claim 16, or a complex of a metal M, wherein said complex comprises a metal ion of said metal M complexed with said bispidine-based metal chelating ligand (I), said metal M being selected from copper, manganese, gallium, cobalt, zinc, nickel, and iron.

31. A nuclear imaging agent for positron emission tomography or single photon emission tomography, said nuclear imaging agent comprising a bispidine-based metal chelating ligand (I) according to claim 16, or a complex of a metal M, wherein said complex comprises a metal ion of said metal M complexed with said bispidine-based metal chelating ligand (I), said metal M being selected from copper, manganese, gallium, cobalt, zinc, nickel, and iron.
